SEQUENCE LISTING (10 sequences)

SEQ ID NO: 1 - DNA, 38 bp, Artificial Sequence
  Description of Artificial SequencePCR amplification primer FVO-PCR1
gggtcggtaccatggcagtaactccttccgtaattgat

SEQ ID NO: 2 - DNA, 48 bp, Artificial Sequence
  Description of Artificial SequencePCR amplification primer FVO-PCR2
ggatcagatgcggccgcttaactgcagaaaataccatcgaaaagtgga

SEQ ID NO: 3 - DNA, 23 bp, Artificial Sequence
  Description of Artificial SequencePCR primer-directed mutagenesis overlapping oligonucleotide EA3
taaaaaatatataaacgacaaac

SEQ ID NO: 4 - DNA, 23 bp, Artificial Sequence
  Description of Artificial SequencePCR primer-directed mutagenesis overlapping oligonucleotide EA5
aaaagggaagatatttctcattt

SEQ ID NO: 5 - DNA, 71 bp, Artificial Sequence
  Description of Artificial Sequencesynthesized sense strand oligonucleotide EA485-CDFVO
cgcagttactccatctgttattgataatattctttctaaaattgaaaacgaatatgaggttttatatttaa

SEQ ID NO: 6 - DNA, 79 bp, Artificial Sequence
  Description of Artificial Sequencesynthesized sense strand oligonucleotide EA493-CDFVO
ggttttaaatataaaacctcatattcgttttcaattttagaaagaatattatcaataacagatggagtaactgcggtac

SEQ ID NO: 7 - DNA, 1065 bp, Plasmodium falciparum
  native codon MSP1-42
gcagtaactccttccgtaattgataacatactttctaaaattgaaaatgaatatgaggttttatatttaaaacctttagcaggtgtttatagaagtttaaaaaaacaattagaaaataacgttatgacatttaatgttaatgttaaggatattttaaattcacgatttaataaacgtgaaaatttcaaaaatgttttagaatcagatttaattccatataaagatttaacatcaagtaattatgttgtcaaagatccatataaatttcttaataaagaaaaaagagataaattcttaagcagttataattatattaaggattcaatagatacggatataaattttgcaaatgatgttcttggatattataaaatattatccgagaaatataaatcagatttagattcaattaaaaaatatataaacgacaaacaaggtgaaaatgagaaataccttccctttttaaacaatattgagaccttatataaaacagttaatgataaaattgatttatttgtaattcatttagaagcaaaagttctaaattatacatatgagaaatcaaacgtagaagttaaaataaaagaacttaattacttaaaaacaattcaagacaaattggcagattttaaaaaaaataacaatttcgttggaattgctgatttatcaacagattataaccataataacttattgacaaagttccttagtacaggtatggtttttgaaaatctcgctaaaaccgttttatctaatttacttgatggaaacttgcaaggtatgttaaacatttcacaacaccaatgcgtaaaaaaacaatgtccacaaaattctggatgtttcagacatttagatgaaagagaagaatgtaaatgtttattaaattacaaacaagaaggtgataaatgtgttgaaaatccaaatcctacttgtaacgaaaataatggtggatgtgatgcagatgccaaatgtaccgaagaagattcaggtagcaacggaaagaaaatcacatgtgaatgtactaaacctgattcttatccacttttcgatggtattttctgcagt

SEQ ID NO: 8 - protein, 355 aa, Plasmodium falciparum
  native codon MSP1-42
AVTPSVIDNILSKIENEYEVLYLKPLAGVYRSLKKQLENNVMTFNVNVKDILNSRFNKRENFKNVLESDLIPYKDLTSSNYVVKDPYKFLNKEKRDKFLSSYNYIKDSIDTDINFANDVLGYYKILSEKYKSDLDSIKKYINDKQGENEKYLPFLNNIETLYKTVNDKIDLFVIHLEAKVLNYTYEKSNVEVKIKELNYLKTIQDKLADFKKNNNFVGIADLSTDYNHNNLLTKFLSTGMVFENLAKTVLSNLLDGNLQGMLNISQHQCVKKQCPQNSGCFRHLDEREECKCLLNYKQEGDKCVENPNPTCNENNGGCDADAKCTEEDSGSNGKKITCECTKPDSYPLFDGIFCS

SEQ ID NO: 9 - DNA, 1065 bp, Artificial Sequence
  Description of Artificial SequencePlasmodium falciparum MSP1-42 with harmonized codons for expression in E. coli cl. 2
gctgttacgccatcggttattgacaatatcctcagcaaaattgaaaacgaatacgaggttctgtacctgaaaccactggctggcgtttaccgttctctgaaaaaacagctggaaaacaatgttatgaccttcaacgttaacgttaaggacattctgaacagccgcttcaacaaacgcgaaaacttcaaaaacgttctggaaagcgacctgattccatacaaagacctgaccagctctaactacgttgtcaaagacccatacaaattcctcaacaaagaaaaacgtgacaaattcctgtcgtcttacaactacattaaggacagcatcgacacggacatcaacttcgctaacgacgttctcggctactacaaaatcctgtcggagaaatacaaaagcgacctggacagcattaaaaaatacatcaatgataaacagggcgaaaacgagaaatatctccctttcctgaataacattgagacgctgtacaaaaccgttaacgacaaaattgacctgttcgttattcacctggaagctaaagttctcaactacacctacgagaaaagcaatgttgaagttaaaatcaaagaactcaactatctgaaaaccattcaggataaactcgctgacttcaaaaaaaacaataacttcgttggcattgctgacctgagcaccgactacaatcacaacaatctgctcaccaagttcctctctaccggcatggttttcgaaaacctcgctaaaacggttctgagcaacctgctcgacggcaatctccagggcatgctgaatattagccagcatcagtgtgttaaaaaacagtgcccacagaacagcggctgcttccgtcacctggacgaacgtgaagaatgcaaatgcctgctgaactataaacaggaaggcgacaaatgcgttgaaaacccaaacccaacgtgcaatgaaaacaacggcggctgcgacgctgacgccaaatgcacggaagaagacagcggctcgaatggcaagaaaattacctgcgaatgcacgaaaccagacagctacccactcttcgacggcattttctgtagt

SEQ ID NO: 10 - protein, 6 aa, Artificial Sequence
  Description of Artificial SequenceN-terminal hexahistidine, His tag, affinity tag
HHHHHH

(19) United States
(12) Patent Application Publication
(10) Pub. No.: US 2004/0209323 A1
(43) Pub. Date: Oct. 21, 2004

(54) Protein expression by codon harmonization and translational attenuation

(75) Inventors: Randall L. Kincaid, Potomac, MD (US); Evelina Angov, Bethesda, MD (US); Jeffrey A. Lyon, Silver Spring, MD (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Veritas

(21) Appl. No.: 10/677,641

(22) Filed: Oct. 1, 2003

Related U.S. Application Data
(60) Provisional application No. 60/425,719, filed on Nov. 12, 2002.

(30) Foreign Application Priority Data
Apr. 1, 2003 (WO) PCT/US03/10384

(51) Int. Cl. C12P 21/02; C12N 1/21; C07H 21/04; C12N 15/74

(52) U.S. Cl. 435/69.1; 435/252.3; 435/471; 435/320.1; 530/350; 536/23.5

(57) ABSTRACT

The present invention is based in the fields of molecular genetics and recombinant protein expression. The invention features compositions and methods for increasing the expression levels of proteins expressed in non-natural hosts. Increased expression levels are achieved by expression of a translationally-harmonized nucleic acid that is created by substituting certain codons having low or intermediate usage frequency in the natural host with synonymous codons that have low or intermediate usage frequency in the non-natural host.

### CROSS-REFERENCE TO RELATED APPLICATIONS

This application claims priority to International Application no. PCT/______, which was filed on Apr. 1, 2003, which application claims the benefit of Provisional Application Serial No. 60/369,741 filed Apr. 1, 2002, Provisional Application Serial No. 60/379,688 filed May 9, 2002 and Provisional Application Serial No. 60/425,719 filed Nov. 12, 2002, all of which are incorporated hereby by reference.

### FIELD OF THE INVENTION

The present invention is based in the fields of molecular genetics and recombinant protein expression. The invention features compositions and methods for increasing the expression levels of proteins expressed in non-natural hosts. Increased expression levels are achieved by expression of a translationally-harmonized nucleic acid that is created by substituting certain codons having low or intermediate usage frequency in the natural host with synonymous codons that have low or intermediate usage frequency in the non-natural host.

### BACKGROUND OF THE INVENTION

Microorganisms, and especially bacteria such as Escherichia coli, are among the most successful vehicles for over-expression of both prokaryotic and eukaryotic proteins. However, expression systems employed to over-express such proteins are not always satisfactory. Under certain conditions of over-expression in bacteria and other non-natural host cells for example, , some heterologous proteins are precipitated within cells as “refractile” or “inclusion” bodies. Such refractile or inclusion bodies consist of dense masses of partially folded, heterologous protein that is often in a form which is not biologically active (S. B. Storrs et al., Protein Folding—American Chemical Society Symposium Series 470, Chapter 15: 197-204, 1991). It is believed that the biological inactivity of refractile or inclusion heterologous proteins is due to incorrect protein folding or assembly brought about , in some instance, by the presence of infrequently used codons in the foreign nucleic acid encoding the protein. These infrequently used codons are believed to cause ribosome pausing leading to incorrect folding and premature termination of protein translation. Consequently, investigators have attempted to overcome translational pausing by replacing infrequently used codons in the foreign nucleic acid with frequently used codons of the host organism. (See, e.g., Robinson et al., Nucleic Acids Res. 12:6663, 1984; U.S. Pat. No. 6,114,148; and 5,786,464). The biological inactivity of refractile or inclusion heterologous proteins due to the process of incorrect protein folding or assembly is believed to occur either before or after intracellular precipitation or during isolation of the proteins.

### SUMMARY OF THE INVENTION

As noted above, previous investigators have attempted to overcome the difficulties inherent in earlier methods of heterologous protein expression by replacing codons in the foreign nucleic acid that are infrequently used in the non-natural host, with codons that are frequently used in the non-natural host; this process is sometimes referred to as “codon optimization”. An important aspect of the present invention is the understanding that it is “harmonization” of the nucleic acid to the non-natural host that is the important consideration when attempting to increase soluble levels of expressed heterologous proteins. In the context of the present invention, harmonization is not simply the replacement of low usage codons with high usage codons, but the identification of codons in the nucleic acid that have low or intermediate codon usage in the natural host, and modifying the nucleic acid to ensure that codons of low or intermediate usage in the non-natural host appear in the same region of the nucleic acid that is to be expressed. Thus, contrary to suggestions of previous publications, the present invention recognizes that codons of low usage frequency in the non-natural host are needed in the nucleic acid to be expressed at positions where codons of low codon usage frequency exist in the native nucleic acid. In other words, to optimize soluble protein expression levels, it is important to focus on codons of low codon usage frequency and ensure that this type of codon is present in the same regions of the nucleic acid being expressed as in the native nucleic acid.

By modifying the nucleic acid to be expressed in this manner, translation of the nucleic acid is selectively attenuated in the non-natural host to match translational procession in the natural host. By allowing the translational machinery to slow or pause at the same positions in the transcript during translation in the non-natural host as in the natural one, secondary structures of the nascent protein are given similar time frames to form in both the non-natural and natural hosts. This allows proteins expressed in non-natural hosts to fold at the rate comparable to that found in the natural host, which increases protein solubility and activity.

Accordingly, the present invention provides a method of increasing soluble levels of a heterologous protein expressed in a non-natural host by selective translational attenuation. The method comprises (a) synthesizing a translationally-harmonized nucleic acid comprising a coding region consisting of at least 60 contiguous codons and having a nucleotide sequence at least 65%, more preferably at least 75%, 85% or, 90%, most preferably 95% or 98% identical to a contiguous codon sequence native to a natural host. One of skill in the art will recognize that, through translational harmonization by synonymous codon exchange, two nucleic acid coding regions can encode the same amino acid sequence while having nucleotide sequence with as little as 65% or less identity. The coding region is translationally-harmonized by substituting each of at least 3, more preferably 6, 8 or 10, most preferably 12, 15, 17, 20 or more codons of the native contiguous codon sequence with a synonymous codon that has a lower usage frequency in the non-natural host. The codons being replaced are (i) within 10, more preferably 8, 6, 4, 2 or 1 codon of a codon in the native codon sequence that has a low or intermediate codon usage frequency in the natural host; and, (ii) of intermediate or high usage frequency in the non-natural host. The harmonized nucleic acid is introduced into the non-natural host cell to generate a non-natural host; and, cultured in the non-natural host under conditions that permit the soluble level of the heterologous protein to exceed by more than 10%, more preferably 20%, advantageously more than 30%, ideally more than 40% over the soluble level of that protein when expressed from a non-harmonized nucleic acid in the non-natural host.

In some aspects of the above method, the translationally-harmonized nucleic acid further comprises expression control sequences operably-linked to the coding region. In other aspects, the heterologous protein is a fusion protein and the translationally-harmonized nucleic acid further comprises a second coding region in-frame with the first coding region. Still other aspects of the invention include a translationally-harmonized nucleic acid that has a nucleotide sequence encoding a peptide linker positioned in-frame with, and located between, the coding region and the second coding region. In some aspects the second coding region of the fusion protein encodes an affinity tag. The second coding region may or may not be translationally-harmonized.

The non-natural host used in aspects of the method can be any cell capable of expressing a harmonized nucleic acid including, bacterial cells, fungal cells, insect cells, mammalian cells and plant cells. In some aspects the natural host is a mammalian tissue cell and the non-natural host is a different mammalian tissue cell. Other aspects of the method include a natural host that is a first cell normally residing in a first mammalian species and the non-natural host is a second cell normally residing in a second mammalian species. In another alternative aspect, the method uses a first cell and the second cell that are from the same tissue type. Exemplary tissue types compatible with the present invention include liver, spleen, lymphoid, smooth muscle, striated muscle, nerve tissue and adipose tissue. In those aspects of the method where the coding region encodes a mammalian protein, the mammalian protein may be a hormone. In other aspects the coding region may encode a neuropeptide, an antibody, an antimetabolites or an antibiotic.

Some aspects of the method also include isolating the heterologous protein. To aid in isolation, the heterologous protein in these aspects may include a secretory sequence. When such secretory sequences are present, the heterologous protein may be isolated by centrifuging the non-natural host culture.

Another embodiment of the present invention is a translationally-harmonized nucleic acid having the characteristics of the nucleic acid constructed using the method discussed above. This translationally-harmonized nucleic acid has all of the features and limitations noted for the nucleic acid constructed using the above method.

Still another embodiment of the invention is a recombinant cell comprising an expression system that includes a translationally-harmonized nucleic acid having the features and limitations of the harmonized nucleic acid noted above. This recombinant cell expresses a soluble level of the heterologous protein exceeding the soluble level of that protein when expressed from a non-harmonized nucleic acid in the recombinant cell by more than 10%, more preferably 20%, advantageously more than 30%, ideally more than 40%.

The invention also includes a computer readable medium comprising (a) code for a first set of instructions for identifying at least three codons in a first set of at least 60 statically ordered codons, wherein the identified codons have a low or intermediate codon usage in a first codon usage data set; and (b) code for a second set of instructions for substituting each of the identified codons with a synonymous codon having a low or intermediate codon usage in a second codon usage data set, thereby generating a second set of at least 60 statically ordered codons. The first set of at least 60 codons is representative of a nucleic acid sequence of a natural host. The first codon usage data set contains data identifying the frequency of codon usage in expressed nucleic acid transcripts of the natural host for each synonymous codon corresponding to each natural amino acid. The second codon usage data set contains data identifying the frequency of codon usage in expressed nucleic acid transcripts of a non-natural host for each synonymous codon corresponding to each natural amino acid. Finally, the second set of at least 60 codons is representative of a nucleic acid translationally-harmonized for expression in the non-native host.

In some aspects of the computer readable medium the first set of instructions identify at least three codons in a first set of at least 60 statically ordered codons. Other aspects of the embodiment harmonize the nucleic acid for expression in a bacterial cell, while other aspects harmonize the nucleic acid for expression in fungal cells, insect cells, plant cells and mammalian cells.

Those aspects that harmonize the nucleic acid for expression in mammals may harmonize a mammalian nucleic acid from a different mammal and/or different tissue. Exemplary tissues for use in these aspects of the invention include liver, spleen, lymphoid, smooth muscle, striated muscle, nerve tissue and adipose tissue. In other aspects the 60 statically ordered codons may encode a mammalian protein such as a mammalian hormone, a neuropeptides, an antibody, an antimetabolites or an antibiotic.

### BRIEF DESCRIPTION OF THE DRAWINGS

FIG. 1 is an Coomassie Blue stained SDS-PAGE gel for Partially Purified Wild type MSP1-42 (FVO) vs. Single Site pause mutant (FMPOO3). Proteins prepared from the soluble fraction of induced cultures of E. coli were subjected to partial purification on NTA-Sepharose and eluate fractions were evaluated by denaturing SDS gel electrophoresis.

FIG. 2 is a Coomassie Blue stained SDS-PAGE gel of Partially Purified MSP1-42 (FVO) (Wild type vs. Single Site pause mutant (FMP003) vs. Initiation Complex harmonized (FMP007)). Proteins prepared from the soluble fraction of induced cultures of E. coli were subjected to partial purification on NTA-Sepharose and eluate fractions were evaluated by denaturing SDS gel electrophoresis.

FIG. 3 is a Coomassie Blue stained SDS-PAGE & Western blot Analysis of lysates from bacteria expressing FMP003, FMP007, or the fully- harmonized gene. Total bacterial lysates were prepared from induced cultures of E. coli and were subjected to denaturing SDS gel electrophoresis (left panel) and Western blot analysis (right panel).

FIG. 4 is a set of Coomassie-stained SDS-PAGE gels comparing lsa-nrc protein expression in E. coli using nucleic acid codon “optimization” and “harmonization”. Whole cell extracts (˜15 ug protein) from uninduced (lanes 1, 3, 5, 7) and induced (lanes 2, 4, 6, 8) cultures were subjected to SDS gel electrophoresis. Coomassie Blue-stained lanes (1, 2, 5, 6) as well as Western blot analysis showed higher amounts of expression with the “harmonized” codon usage than with “optimized” codon usage.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

“Natural host” refers to a cell that is expressing a protein from a nucleic acid transcript that is naturally present in the host.

A molecule, more particularly for the present invention a protein or nucleic acid is “native to a natural host” when the molecule is normally part of the natural host in the wild, and is not introduced to the host through molecular biological techniques.

“Non-natural host” refers to a cell that is expressing a protein from a nucleic acid transcript that is not naturally found in the natural host, as occurs in recombinant cells formed through molecular biological techniques.

“Coding region” refers to the portion of a nucleic acid transcript or gene that can be translated into polypeptide or protein when operably linked to expression control sequences and introduced to a suitable translation system.

“Translation system” refers to an enzyme-based system capable of producing a protein de novo when a nucleic acid transcript is introduced into the system.

“Contiguous codons” refers to two or more codons linked in frame with each other without any intervening nucleotides.

“Synonymous codons” are sets of codons whose corresponding tRNAs are charged with a common amino acid such that, when appearing in a transcript, the amino acid positions corresponding to synonymous codons are occupied by the same amino acid.

“Selective translational attenuation” refers to a process by which certain codons of a nucleic acid transcript are substituted with synonymous codons in order to affect the rate of protein translation.

The “codon usage frequency” for a cell or organism is calculated by determining the frequency at which a given synonymous codon is used in cellular transcripts relative to other synonymous codons corresponding to the same amino acid. Codon usage frequency is defined as the “percentage usage of a given synonymous codon within that set of synonymous codons”. For instance, the global usage frequencies in Homo sapiens for the four codons encoding the amino acid valine (GTT, GTC, GTA and GTG), are 11, 14.6, 7.2 and 28.4 codons, respectively, per 1000 codons in all human transcripts. By summing the global usage frequencies for these four codons (a total of 48.2 per 1000) the individual codon usage frequencies can be obtained simply by divided this value by that seen for a specific codon. Therefore, within this set of synonymous codons, the usage frequencies calculated for each codon are 18%, 23.9%, 11.8% and 46.4%, respectively.

To more easily compare usage frequencies between different sets of synonymous codons, the term “relative codon usage” can be applied, which takes into account the differing number of members in sets of synonymous codons (e.g., 2, 4 or 6 codons); this comparative value has also been called “relative synonymous codon usage” or RSCU (Sharp, P. M., and W. H. Li, (1987). Nucleic Acids Research 15: 1281-1295). To calculate this value, one divides the codon usage frequency for an individual codon (converted to a percentage—0.18=18%) by the arithmetic mean value (as a simple fraction) for that set of codons and then subtracts the value of 100; for the valine codon GTT in the example above, this would be (18/0.25)−100 or −28%. Relative codon usage, then, is the “percentage deviation from the mean” for a given codon group, i.e., if the arithmetic mean usage for valine (4 codons) is 25% (zero deviation), then a codon usage frequency of 18% for a valine codon (GTT, in the example above) would represent a negative deviation from the mean of 28% or a relative codon usage of −28%; likewise, a codon usage frequency of 46% for a valine codon (GTG, in the example above) would represent a positive deviation from the mean of 86% or a relative codon usage of +86%.

A “translationally-harmonized nucleic acid” or “harmonized nucleic acid” is any nucleic acid that encodes a protein and has been modified to improve soluble levels of the protein when the nucleic acid is expressed in a non-natural host by at least 10%, more preferably 20%, advantageously more than 30%, ideally more than 40% over the levels achieved when expressing the unmodified nucleic acid in the non-natural host under the same conditions, where the modifications are substitutions of selected codons with synonymous codons to achieve selective translational attenuation.

A “non-harmonized nucleic acid” is any nucleic acid that encodes a protein and has not been modified to improve soluble levels of the protein when the nucleic acid is expressed in a non-natural host, where the modifications are substitutions of selected codons with synonymous codons to achieve selective translational attenuation.

“Expression control sequences” are those nucleotide sequences, both 5′ and 3′ to a coding region, that are required for the transcription and translation of the coding region in a host organism. Regulatory sequences include a promoter, ribosome binding site, optional inducible elements and sequence elements required for efficient 3′ processing, including polyadenylation. When the structural gene has been isolated from genomic DNA, the regulatory sequences also include those intronic sequences required for splicing of the introns as part of mRNA formation in the target host.

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2′-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

Amino acids may be referred to herein by either commonly known three letter symbols or by the one-letter symbols recommended by the IUPAC-IUB Biochemical Nomenclature Commission. Nucleotides, likewise, may be referred to by their commonly accepted single-letter codes.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, 65%, 70%, 75%, 80%, preferably 85%, 90%, 91%, 92%, 93%, 94%, 95%, 96%, 97%, 98%, 99% or higher identity to a reference sequence), when compared and aligned for maximum correspondence over a comparison window, or designated region as measured using one of the following sequence comparison algorithms or by manual alignment and visual inspection.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of translationally-harmonized nucleic acid sequences and the polypeptides they encode, the BLAST and BLAST 2.0 algorithms and the default parameters discussed below are used.

A “comparison window”, as used herein, includes reference to a segment of any one of the number of contiguous positions in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

“Secretory sequence” refers to a nucleotide sequence encoding an amino acid sequence that, when appearing in a protein, directs the cellular machinery to export the protein containing the amino acid sequence to the cell exterior

“Expression system” refers to the combination of expression control sequences that, when operably linked to a coding region, allows the coding region to be transcribed into protein when placed in a suitable translation system.

The term “operably linked” refers to a linkage of polynucleotide elements in a functional relationship. With regard to the present invention, the term “operably linked” refers to a functional linkage between a nucleic acid expression control sequence (such as a promoter, or a ribosome binding site) and a second nucleic acid sequence, e.g., wherein the expression control sequence directs or initiates translation of the nucleic acid corresponding to the second sequence and, in some instances, itself. Thus, a nucleic acid is “operably linked” when it is placed into a functional relationship with another nucleic acid sequence.

A “peptide linker” is a series of at least 3, preferably at least 6, more preferably at least 10 or more amino acids that covalently couples two polypeptides or proteins together.

“Fusion protein” refers to a protein formed from the non-natural coupling of two polypeptides or proteins. The fusion protein is created from a recombinant nucleic acid that includes coding regions for the two polypeptides linked in frame with each other. The nucleic acid may also include a coding region for a peptide linker located between, and in frame with, the coding regions for the two polypeptides.

“Affinity tag” refers to an amino acid sequence that may be specifically recognized by another molecule or molecular complex. Exemplary affinity tags include epitope tags, FLAG tags and His-tag sequences.

### DETAILED DESCRIPTION

The present invention overcomes the problem of expressing high levels of soluble recombinant proteins by providing methods and compositions that include harmonized nucleic acids encoding the desired recombinant proteins. This is accomplished by modifying a native nucleic acid encoding the desired protein by substituting codons having a low or intermediate codon usage frequency in a natural host, with a synonymous codon having a similar codon usage frequency in a non-natural host that will serve as an translation system for the modified nucleic acid. The modified nucleic acid is termed a “translationally-harmonized nucleic acid” that is designed to express soluble levels of the encoded protein that are at least 10% higher than the levels expressed using the unmodified nucleic acid in a non-natural host.

Before the nucleic acid can be harmonized, the codon usage frequency for each codon corresponding to an amino acid in the recombinant protein is determined. The codon usage frequency in the native host is determined for each codon present in the native nucleic acid encoding the desired protein. Codon usage frequencies in the selected non-native host are also determined for all codons corresponding to amino acids of the desired protein. Each codon is then categorized as being of low, intermediate or high codon usage frequency, as described below.

Once codon usage frequencies have been determined and categorized, the nucleic acid encoding the desired protein can be harmonized for expression in the non-native host. Harmonization of the nucleic acid is accomplished by ensuring that amino acids encoded by codons that have a given codon usage frequency category in the native host will also be encoded by codons of the same codon usage frequency category in the non-native host. As codon usage frequency may differ from organism to organism, or even between tissues of the same organism, ensuring that a nucleic acid is harmonized for expression in the selected non-native host may require substituting codons of the native nucleic acid sequence with synonymous codons. Appropriate synonymous codons will be of the same category of codon usage frequency in the non-native host as the native codon they replace has in the native host. By making synonymous codon substitutions in the native nucleic acid in this way, a harmonized nucleic acid is created that, when introduced to the non-native host, may be expressed with the same translational attenuation as found for the native nucleic acid in the native host.

Harmonized nucleic acids of the present invention find utility in a variety of areas including the expression of therapeutically and economically important proteins, and as part of gene therapy regimes.

I. Determining Codon Usage Frequency

As noted above, determination of the codon usage frequency in the native host for each codon present in the native nucleic acid sequence is a prerequisite to harmonizing the coding sequence. Similarly, the codon usage frequency in the non-native host for synonymous codons of the codons identified in the native nucleic acid sequence need to be identified. This section discusses the methods of determining codon usage, and the limits and conditions considered when categorizing codon usage frequency in detail.

A. Calculating Codon Usage Frequency

The codon usage frequency for a cell or organism is calculated by determining the frequency where a given synonymous codon is used in cellular transcripts relative to other synonymous codons corresponding to the same amino acid. Codon usage frequency is defined as the “percentage usage of a given synonymous codon within that set of synonymous codons”. For instance, the global usage frequencies in Homo sapiens for the four codons encoding the amino acid valine (GTT, GTC, GTA and GTG), are 11, 14.6, 7.2 and 28.4 codons, respectively, per 1000 codons in all human transcripts. By summing the global usage frequencies for these four codons (a total of 48.2 per 1000) the individual codon usage frequencies can be obtained simply by divided this value by that seen for a specific codon. Therefore, within this set of synonymous codons, the usage frequencies calculated for each codon are 18%, 23.9%, 11.8% and 46.4%, respectively. To more easily compare usage frequencies between different sets of synonymous codons, the term “relative codon usage” can be applied, which takes into account the differing number of members in sets of synonymous codons (e.g., 2, 4 or 6 codons); this comparative value has also been called “relative synonymous codon usage” or RSCU (Sharp, P. M., and W. H. Li, (1987). Nucleic Acids Research 15: 1281-1295). To calculate this value, one divides the codon usage frequency for an individual codon (converted to a percentage—0.18=18%) by the arithmetic mean value (as a simple fraction) for that set of codons and then subtracts the value of 100; for the valine codon GTT in the example above, this would be (18/0.25)−100 or −28%. Relative codon usage, then, is the “percentage deviation from the mean” for a given codon group, i.e., if the arithmetic mean usage for valine (4 codons) is 25% (zero deviation), then a codon usage frequency of 18% for a valine codon (GTT, in the example above) would represent a negative deviation from the mean of 28% or a relative codon usage of −28%; likewise, a codon usage frequency of 46% for a valine codon (GTG, in the example above) would represent a positive deviation from the mean of 86% or a relative codon usage of +86%.

The global codon usage data used to calculate usage frequencies, are preferably those posted on the Kazusa Codon Usage Database (See the www website kazusa.orjp/codon/; and Nakamura, Y., Gojobori, T. and Ikemura, T. (2000) Nucl. Acids Res. 28, 292). However, these data may be refined by injecting other considerations relating to the desired protein or amino acid. For example, the subset of transcripts from which the codon usage frequency is calculated can be limited to transcripts encoding proteins similar to, or produced under conditions similar to, the desired protein. In preferred embodiments, the codon usage frequencies for non-natural hosts used with the present invention are calculated using a dataset consisting of transcripts of proteins (e.g., ribosomal proteins) that are highly and continuously expressed during the growth phase, i.e., under the growth conditions that will be used when expressing the translationally-harmonized nucleic acid in the non-natural host. In E. coli, this set of transcripts correspond to so-called class II proteins (form Henaut and Danchin: Analysis and Predictions from Escherichia coli sequences in: Escherichia coli and Salmonella, Vol. 2, Ch. 114:2047-2066, 1996, Neidhardt F C ed., ASM press, Washington, D.C.)

B. Categorizing Relative Codon Usage Frequencies.

Once the relative codon usage frequencies have been calculated, each codon may be categorized as being of high, intermediate or low codon usage frequency. For purposes of the present invention low frequency codon usage are those codons used in a sample data set of nucleic acid transcripts with a relative codon usage (deviation from the mean usage value for that codon set) of less than or equal to −33%. Codons of intermediate codon usage have relative codon usage values between −33% and 25%, while high usage frequency codons display a relative codon usage value that is greater than 25%.

II. Producing Translationally-Harmonized Nucleic Acids

Once the codon usage frequency tables (including the derived relative codon usage values) for the native and the non-native hosts have been determined, a nucleic acid from the natural host encoding the desired heterologous protein may be translationally-harmonized for expression in the non-natural host. The first step in the process involves identifying codons in the natural nucleic acid that are of low or intermediate codon usage frequency in the natural host. These codons are then substituted with codons of similar low or intermediate codon usage frequency in the non- natural host. If a harmonization “choice” is required (i.e., to select between two codons in the non-natural host having similar relative codon usage values), the codon with a lower relative codon usage value is selected, unless that value is—90% or lower. Typically at least 3, more preferably 6, 8 or 10, most preferably 12, 15, 17, 20 or more codons of the natural nucleic acid sequence are substituted in this manner. After translational harmonization by substitution of synonymous codons, the harmonized nucleic acid will be at least 65%, more preferably at least 75%, 85% or, 90%, most preferably 95 or 98% identical to the at least 60 contiguous codons of the native nucleic acid sequence encoding the desired heterologous protein.

In some instances, there may not be a synonymous non- natural host codon of suitable low or intermediate codon usage frequency available to substitute for an identified natural codon; this may arise due to evolutionary biases in synonymous codon in different species. In such situations, a neighboring codon may be substituted with a synonymous codon in lieu of the identified codon. For example, using the teachings provided in the present invention, one of skill will recognize that harmonization of nucleic acid sequence, and the desired translational attenuation, may be achieved by substituting a neighboring codon having a high codon usage frequency in both the natural and non-natural hosts with a synonymous codon having a low or intermediate codon usage frequency in the non- natural host, provided that the neighboring codon being substituted is within 10 preferably within 8, 6 or 4, more preferably within 3, 2 or 1 codon of the identified native codon.

In other instances, the natural host codon will have a codon usage frequency that is nearest in value to a non-natural host synonymous codon in the high codon usage frequency category. This may happen, for example, where there is a wide range of codon usage frequencies for a non-natural host synonymous codon set. In such instances, based on the teachings presented herein, one of skill in the art will recognize that translational harmonization compels the use of the synonymous non-natural host codon having the closest codon usage frequency to that of the natural codon which is less than the codon usage frequency of the natural codon. This rule follows the general thesis of the invention that, by default, choosing synonymous non-natural host codons having a lower codon usage frequency than the natural codon are preferred. Thus if there is no non-natural host synonymous codon with a codon usage frequency that differs from that of the natural codon by less than 25%, preferably less than 15%, most preferably less than 5% of the absolute value of the relative codon usage frequency of the natural codon, then the synonymous non-natural host codon having a codon usage frequency nearest to but less than the natural codon usage frequency is substituted for the natural codon.

However, the natural codon should never be replaced with a non-natural host codon having a codon usage frequency that differs from that of the natural codon by more than 5%, preferably not more than 15%, most preferably not more than 25% of the absolute value of the relative codon usage frequency of the natural codon that also has a relative codon usage frequency of less than −90. One of skill in the art will recognize that replacement of the natural codon with such a rarely used synonymous codon creates a danger of excessive translational attenuation with the possibility of premature translational termination.

Substitution of a codon with a synonymous codon may be performed using any suitable method known to those of skill in the art. Exemplary methods for performing such substitutions are described below.

A. Translational Harmonization Using Computer-Generated Nucleotide Sequences

While determining which codons may be substituted to achieve translational harmonization may be done manually, the process lends itself to automation and is preferably performed using a computer system with instructions written on a computer-readable media. For example, the computer system may be used to create codon usage tables for natural and non-natural hosts. This may be accomplished by supplying data sets of the coding regions taken from the transcripts for expressed proteins from the natural and non-natural hosts. Each of the data sets is surveyed to determine the codon usage for each codon present in the data set. The results may then be tabulated, forming a codon usage tables for the natural and non-natural hosts.

The generated codon usage tables may then be used to identify codons of low or intermediate codon usage in a natural host nucleotide sequence that may require substitution with a synonymous codon to harmonize the nucleotide sequence for expression in the non-natural host. For example the computer system may include code for a first set of instructions for identifying at least three codons, in a first set of at least 60 statically ordered codons, that have a low or intermediate codon usage in the natural host codon usage data set. Code is also supplied for determining the relative codon usage frequency in the non-natural host for each of the identified codons, using the second codon usage data set. If the identified codon has a low or intermediate relative codon usage in the non-natural host that differs from that of the natural host by not more than 25%, preferably not more than 15%, most preferably not more than 5% the codon usage frequency in the natural host, then there is no need to substitute the natural codon. One of skill in the art will recognize that in such situations where the codon usage frequencies for an identified codon in the natural and non-natural host are similar, a synonymous codon need not be substituted to translationally-harmonize the nucleic acid.

If however the non-natural host codon usage frequency of the identified codon is found differ by more than 25% from the natural host codon usage frequency for the codon, then the computer system will select a synonymous codon from the second codon usage data set that has a codon usage frequency that is not more than 25%, preferably not more than 15%, most preferably not more than 5% the codon usage frequency for the identified codon in the natural host. If there is no suitable synonymous codon that can be substituted at the position of the identified codon, the computer system has code that will check the suitability for substitution of alternative codons either side of the identified codon. An suitable alternative codon is within 10, preferably within 8, 6 or 4, more preferably within 3, 2 or 1 codon of the identified codon, and has a high codon usage frequency in the natural host. If the alternative codon also has a high codon usage frequency in the non-natural host, the computer system will consult the second codon usage data set for a synonymous codon having a low or intermediate codon usage in the non-natural host. One of skill in the art will recognize that the presence of an alternative codon that coincidentally has a low or intermediate codon usage frequency in the non-natural host will negate the need for substituting a synonymous codon for an alternative codon in the region of the identified codon. Accordingly, the computer system may be programmed to identify and adjust to such a situation.

B. Synthesizing and Modifying Nucleic Acids

Translationally-harmonized nucleic acids may be constructed from nucleic acids isolated from natural sources using techniques well known to those of ordinary skill in the art (See, e.g., Sambrook, J., Fritsch, E. F., and Maniatus, T., Molecular Cloning, A Laboratory Manual 2nd ed. (1989). For example, the nucleic acid to be translationally harmonized may be isolated from a natural source, such as a tissue or cellular source, known to express the protein encoded by the nucleic acid. Generally, cDNA or genomic libraries are constructed and screened to identify the correct sequence. (For cDNA libraries, see e.g., Gubler & Hoffman, Gene, 25:263-269 (1983); Sambrook et al., 2001, Molecular Cloning: A Laboratory Manual (3^{rd} ed.); Cold Spring Harbor Laboratory, Cold Spring Harbor, N.Y.; Ausubel et al. (eds.), 1993, Current Protocols in Molecular Biology, John Wiley & Sons, NY. For genomic libraries, see Benton & Davis, Science, 196:180-182 (1977); Grunstein et al., Proc. Natl. Acad. Sci. USA., 72:3961-3965 (1975); and Gussow, D. and Clackson, T., Nucl. Acids Res., 17:4000 (1989).)

Alternatively, nucleic acids of the invention may be synthesized chemically using solution or solid phase techniques well known by those of skill in the art, for example using the solid phase phosphoramidite triester method described by Beaucage and Caruthers (1981), Tetrahedron Letts., 22(20):1859-1862, e.g., using an automated synthesizer, as described in Needham-VanDevanter et al. (1984) Nucleic Acids Res., 12:6159-6168. Oligonucleotides can also be custom made or ordered from a variety of commercial sources known to persons of skill in the art. Purification of oligonucleotides, where necessary, is typically performed by either native acrylamide gel electrophoresis or by anion-exchange HPLC as described in Pearson and Regnier (1983) J. Chrom. 255:137-149.

Chemical synthesis of oligonucleotides encoding dsRNA's can also be performed using nucleotide analogs. Use of analogs frequently confers desirable properties to the oligonucleotide, such as resistance to nucleases, or ease of entry into cells during transformation. Preferred nucleotide analogs are unmodified G, A, T, C and U nucleotides; pyrimidine analogs with lower alkyl, alkynyl or alkenyl groups in the 5 position of the base and purine analogs with similar groups in the 7 or 8 position of the base. Other preferred nucleotide analogs are 5-methylcytosine, 5-methyluracil, diaminopurine, and nucleotides with a 2′-O-methylribose moiety in place of ribose or deoxyribose. As used herein lower alkyl, lower alkynyl and lower alkenyl contain from 1 to 6 carbon atoms and can be straight chain or branched. These groups include methyl, ethyl, propyl, isopropyl, butyl, isobutyl, tertiary butyl, amyl, hexyl and the like. A preferred alkyl group is methyl.

Nucleic acids isolated from native sources may be harmonized for expression in a non-native host using methods well-known in the art including site-specific mutagenesis and other well-known techniques. See, e.g., Berger and Kimmel, Guide to Molecular Cloning Techniques, Methods in Enzymology, Volume 152 Academic Press, Inc., San Diego, Calif. (Berger); Sambrook et al., Molecular Cloning—A Laboratory Manual (2nd ed.) Vol. 1-3, Cold Spring Harbor Laboratory, Cold Spring Harbor Press, N.Y., (Sambrook) (1989); and Current Protocols in Molecular Biology, F. M. Ausubel et al., eds., Current Protocols, a joint venture between Greene Publishing Associates, Inc. and John Wiley & Sons, Inc., (1994 Supplement) (Ausubel); Pirrung et al., U.S. Pat. No. 5,143,854; and Fodor et al., Science, 251:767-77 (1991). Using these techniques, it is possible to insert or delete, at will, a polynucleotide of any length into a DNA expression cassette described herein.

Site-directed mutagenesis techniques are described in Ling et al., “Approaches to DNA mutagenesis: an overview”, Anal Biochem., 254(2):157-178 (1997); Dale et al., “In vitro mutagenesis”, Ann. Rev. Genet., 19:423-462 (1996); Botstein & Shortle, “Strategies and applications of in vitro mutagenesis”, Science, 229:1193-1201 (1985); Carter, “Site-directed mutagenesis”, Biochem. J, 237:1-7 (1986); and Kunkel, “The efficiency of oligonucleotide directed mutagenesis” in Nucleic Acids & Molecular Biology (Eckstein, F. and Lilley, D. M. J. eds., Springer Verlag, Berlin) (1987)); mutagenesis using uracil containing templates (Kunkel, “Rapid and efficient site-specific mutagenesis without phenotypic selection”, Proc. Natl. Acad. Sci. USA, 82:488-492 (1985); Kunkel et al., “Rapid and efficient site-specific mutagenesis without phenotypic selection”, Methods in Enzymol., 154:367-382 (1987); and Bass et al. (1988); oligonucleotide-directed mutagenesis (Methods in Enzymol., 100:468-500 (1983); Methods in Enzymol., 154:329-350 (1987); Zoller & Smith, “Oligonucleotide-directed mutagenesis using M13-derived vectors: an efficient and general procedure for the production of point mutations in any DNA fragment”, Nucleic Acids Res., 10:6487-6500 (1982); Zoller & Smith “Oligonucleotide-directed mutagenesis of DNA fragments cloned into M13 vectors”, Methods in Enzymol., 100:468-500 (1983); and Zoller & Smith, “Oligonucleotide-directed mutagenesis: a simple method using two oligonucleotide primers and a single-stranded DNA template”, Methods in Enzymol., 154:329-350 (1987)); Taylor et al. (1985) “The rapid generation of oligonucleotide-directed mutations at high frequency using phosphorothioate-modified DNA”, Nucl. Acids Res., 13: 8765-8787 (1985); Nakamaye & Eckstein, “Inhibition of restriction endonuclease Nci I cleavage by phosphorothioate groups and its application to oligonucleotide-directed mutagenesis”, Nucl. Acids Res., 14:9679-9698 (1986); Sayers et al., “Y-T Exonucleases in phosphorothioate-based oligonucleotide-directed mutagenesis”, Nuc. Acids Res., 16:791-802 (1988); and Sayers et al. (1988); mutagenesis using gapped duplex DNA (Kramer et al., “The gapped duplex DNA approach to oligonucleotide-directed mutation construction”, Nuc. Acids Res., 12:9441-9456 (1984); Kramer & Fritz, “Oligonucleotide-directed construction of mutations via gapped duplex DNA”, Methods in Enzymol., 154:350-367 (1987); Kramer et al., “Improved enzymatic in vitro reactions in the gapped duplex DNA approach to oligonucleotide-directed construction of mutations”, Nucl. Acids Res., 16:7207 (1988); and Fritz et al., “Oligonucleotide-directed construction of mutations: a gapped duplex DNA procedure without enzymatic reactions in vitro”, Nucl. Acids Res., 16:6987-6999 (1988)).

Oligonucleotides may be purified by polyacrylamide gel electrophoresis, or by any of a number of chromatographic methods, including gel chromatography and high-pressure liquid chromatography. The sequence of the synthetic oligonucleotides can be verified using the chemical degradation method of Maxam and Gilbert (1980) in Grossman and Moldave (eds.) Academic Press, New York, Methods in Enzymology 65:499-560. For nucleic acids, sizes are given in either kilobases (Kb) or base pairs (bp). These are estimates derived from agarose or acrylamide gel electrophoresis, from sequenced nucleic acids, or from published DNA sequences.

C. Proteins that may be Expressed from Harmonized Nucleic Acids

Any protein capable of being expressed in a prokaryotic and/or eukaryotic cell may be encoded by and expressed from a translationally harmonized nucleic acid of the present invention. Such proteins include those from mammalian, insect, plant, fungal, and avian sources, or other sources expressing the desirable protein that are also amenable to extraction of the nucleic acid(s) encoding the desirable protein using techniques known to those of skill in the art. Typical classes of proteins expressed from translationally harmonized nucleic acids include neuropeptides, antibodies, antimetabolites, peptidic hormones, growth factors, chemotactic and chemotropic agents, antibiotics, candidates for therapeutic vaccines and the like.

The nucleic acids of the invention are structurally characterized as comprising at least one coding region having at least 60, more preferably at least 65, 70 or 75, desirably 80, 85, or 90 or more contiguous codons encoding the desirable protein. Nucleic acids of the present invention have at least 3, preferably at least 6, 8, 10 or 12, most preferably at least 15, 17, 20 or more codons that are synonymous to, but different from those present in the native nucleic acid sequence. Thus after translational harmonization, nucleic acids of the invention have a nucleotide sequence that is at least 65%, more preferably 75%, 85% or 90%, desirably 95% or 98% identical to the native nucleic acid sequence, and encode the same protein as the native nucleic acid.

Fusion Proteins

Preferred embodiments of the present invention include fusion proteins. For purposes of the present invention, fusion proteins are heterologous molecules that do not occur naturally in nature, and are formed from two or more native amino acid sequences that are coupled either directly or via a linker molecule.

Typically, a fusion protein is formed by expressing a fusion gene encoding the fusion protein. The fusion gene comprises nucleic acid sequences encoding the amino acid sequences of the fusion protein that are operably linked such that the codons of the respective nucleic acids are in-frame with each other. For purposes of the present invention, at least one of the coding nucleic acids being linked comprises at least 65, 70 or 75, desirably 80, 85, or 90 or more contiguous codons. Other nucleic acids of the fusion protein may have any number of codons within their sequence, but preferably are also coding sequences having at least 65, 70 or 75, desirably 80, 85, or 90 or more contiguous codons.

The coding nucleic acids forming the fusion protein may be directly coupled inframe to one another, or may be indirectly coupled via an intervening nucleic acid encoding a linker sequence. Linkers are preferably polypeptides of between 6 and 28 amino acids in length although longer linker sequences are also contemplated as being part of the invention. The linkers joining the two molecules are preferably designed to;

(1) allow the two molecules to fold and act independently of each other,

(2) not have a propensity for developing an ordered secondary structure which could interfere with the functional subunits of the two proteins, and

(3) have minimal hydrophobic or charged characteristic which could interact with the functional protein subunits

Amino acids used in linkers are preferably found in flexible protein regions. Exemplary amino acids of this type include Gly, Asn and Ser. Virtually any permutation of amino acid sequence containing Gly, Asn and Ser would be expected to satisfy the above criteria for a linker sequence. Neutral amino acids, such as Thr and Ala, may also be used in the linker sequence. Preferably such amino acids will have a relatively small surface area (160 Å², or less). Additional amino acids may also be included in the linkers due to the addition of unique restriction sites to facilitate construction of the fusions, as will be appreciated by those of skill in the art. The present invention is however, not limited by the form, size, composition or number of amino acids employed. The only requirement of the linker is that, functionally, it does not interfere adversely with the folding and function of the individual amino acid sequences of the fusion protein, and otherwise allows for efficient expression and folding of the fusion molecule.

The present invention also includes linkers in which an endopeptidase recognition sequence is included. Such cleavage sites may be valuable to separate the individual components of the fusion to, for example, determine if they are properly folded and active in vitro. Exemplary endopeptidases include, but are not limited to, Plasmin, Enterokinase, Kallikrein, Urokinase, Tissue Plasminogen activator, clostripain, Chymosin, Collagenase, Russell's Viper Venom Protease, Postproline cleavage enzyme, V8 protease, Thrombin and factor Xa.

Particularly preferred fusion proteins include amino acid sequences that facilitate purification or identification of the fusion protein. Exemplary amino acid sequences of this type include affinity tags and secretory sequences. Other tags or labels are also known in the art and applicable to the present invention. Common fusion protein sequences of this type include glutathione S-transferase (“GST”), thioredoxin (“Trx”), maltose binding protein, C- and/or N-terminal hexahistidine polypeptide (His tag), polylysine and other binding molecules. Other embodiments are coupled to elements that allow the fusion proteins to be easily identified, such as small fluorescent proteins, antigenic determinants(e.g., FLAG, CD4, HA), enzymes that produce detectable products and the like. Still other embodiments are coupled to signal elements that direct the target products to particular cellular compartments. Examples of signal elements include those directing proteins to cellular organelles or identify the protein for excretion, the secretory signal segments.

Preferred fusion protein embodiments of the present invention include a coding region for a secretory signal segment. Secretory signal segments are typically N-terminal amino acid sequences capable of directing a polypeptide into the secretory pathway characteristic of eukaryotic cells. As these N-terminal amino acid sequences are typically cleaved as part of the secretory process, secretory signal segments useful in the practice of the present invention can easily be identified. For example the N-terminal amino acid sequence of a secreted protein can be compared with the amino acid sequence predicted from the cDNA sequence encoding the same protein. The N-terminal amino acids predicted by the cDNA sequence but missing from the excreted protein constitute a prospective signal sequence. A nucleic acid encoding this prospective signal sequence is potentially a secretory signal segment.

The prospective secretory signal segment can be tested for functionality by ligating it in-frame to a reporter gene, such as the coding sequence for alkaline phosphatase or green fluorescent protein. The resulting chimeric protein is then inserted into a suitable expression vector and transfected into a host cell where it can be expressed. Expression of the chimeric protein leading to appearance of the reporter gene product in the extracellular fluid indicates that the secretory signal segment is functional. Transmembrane domains may also be incorporated into fusion protein construct to link otherwise secreted proteins to the cell surface.

D. Determining the Degree of Identity of Harmonized Nucleic Acids with Native Nucleic Acids

For purposes of the present invention, the translationally-harmonized nucleic acid sequence is at least 65%, more preferably 75%, 85% or 90%, desirably 95% or 98% identical to the native nucleic acid sequence, and encodes the same protein as the native nucleic acid. The nucleotide sequences of the respective nucleic acids may be sequenced using any method known in the art, for example sequencing may be performed using the solid phase phosphoramidite triester method described by Beaucage and Caruthers (1981), Tetrahedron Letts., 22(20):1859-1862, e.g., using an automated synthesizer, as described in Needham-VanDevanter et al. (1984) Nucleic Acids Res., 12:6159-6168. The sequences may then, for example, be compared manually or with the aid of a computer system to determine the percentage sequence identity between the two nucleic acids over the at least 60 contiguous codons encoding the desired protein. Suitable computer systems for determining sequence identity are well-known in the art, for example the NBLAST system discussed supra.

Another characteristic of harmonized nucleic acids of the present invention is that they preferably encode the same protein as the natural nucleic acid isolated from the natural host. Ideally, the proteins expressed from the respective nucleic acids are identical in amino acid sequence. Some conserved substitutions of non-critical amino acids with alternative amino acids having similar chemical and/or structural properties is however permissible, where the substitution is necessary or desirable in achieving the desired protein. The ability to make such conservative substitutions is well known to those of skill in the art and can be performed with routine experimentation with the benefit of the disclosures found in the present invention.

Confirmation that the respective nucleic acids encode the same protein may be done by any suitable method known in the art. For example, the amino acid sequence of the encoded protein may be determined indirectly from the nucleic acid itself; by automated sequencing of peptides proteolytically-generated from the expressed protein; or mass spectrometric analysis. Data generated from proteins expressed from the native and the translationally-harmonized nucleic acids can then be compared using methods well-known to those of skill in the art. Alternatively, samples of the respective expressed proteins can simply be compared using peptide mapping techniques, such as those described in Gibson, W. (1974) Virology. 62(2):319-36; Beemon, K., and Hunter, T. (1978) J. Virol. 28(2):551-66; and Luo, K., Hurley, T. R., and Sefton, B. M. (1990) Oncogene 5(6):921-3.

III. Expressing Translationally-Harmonized Nucleic Acids

Expressing desired proteins from translationally-harmonized nucleic acids of the present invention requires that the translationally-harmonized coding region be operably-linked with suitable expression control sequences that allow the coding region to be translated when introduced to a competent translation system. Expression control sequences minimally include promoter and termination sequences. Optional control sequences may also be used, including transcriptional and translational control sequences such as enhancers or repressors.

Suitable expression control sequences are frequently application-dependent and are selected based on the translation system to be used. For example, if the translation system is a cell the expression control sequences operably-linked to the harmonized nucleic acid coding sequence are necessarily selected from those that are functional in the cell, preferably native to the cell.

Preferably, the expression control sequences are part of a plasmid or viral genome expression system that aids in introducing the translationally-harmonized nucleic acid to the translation system, preferably a non-natural host cell, in a form that allows expression of the protein encoded by the harmonized nucleic acid.

Expression systems suitable for use with the present invention may be from any source, provided their function allows translation of the translationally-harmonized nucleic acids of the present invention in the chosen translation system. Preferably all of the components of the chosen expression system are from the same source, more preferably from the same biological source, where their function in a chosen non-natural host cell has been characterized or can be confidently predicted by one of skill in the art. Suitable sources for expression system components include genetic sequences of the non-natural host cell itself, viruses, bacteria and the like. Expression systems suitable for use in the present invention can be constructed by those of skill in the art through routine experimentation using knowledge commonly known by those of skill. Suitable expression systems are also available through commercial vendors such as, for example, Vector Laboratories Inc., InVitrogen, Promega, Novagen, NEB, Clontech, Boehringer Mannheim, Pharmacia, EpiCenter, OriGenes Technologies Inc., Stratagene, PerkinElmer, Pharmingen, Life Technologies, Inc., and Research Genetics. Preferably expression systems are formed from the non-natural host from which the translationally-harmonized nucleic acid is to be expressed, more preferably the expression system is formed from components of a transcript that is expressed during the exponential log growth phase of the non-natural host.

Nucleic acids of the present invention may be circular or linear in structure. When present in a cell-based translation system, the nucleic acids may be, integrated into the host cell chromosome or present in the cell epigenetically; stably passed to daughter cells or present transiently in the cell; located anywhere within the cell compatible with expression of the encoded protein, which for eukaryotic host cells includes the cytoplasm, nucleus, and/or an organelle. Nucleic acids including expression systems of the invention may also include origins of replication, restriction sites (both naturally-occurring and engineered), sequences encoding selectable or scorable markers or any other useful genetic element including those that aid in identifying correctly formed expression systems, recombinant cells, controlling harmonized nucleic acid expression, and isolating protein products expressed from translationally-harmonized nucleic acids of the present invention.

B. Suitable Non-Natural Host Cells

Preferred translation systems for expressing the translationally harmonized nucleic acids of the present invention are cell-based, comprising a non-natural host cell. Non-natural host cells suitable for use in the present invention include both prokaryotic and eukaryotic cells. Preferred non-natural prokaryotic host cells include gram positive bacteria, for example a Bacillus cell, e.g., Bacillus alkalophilus, Bacillus amyloliquefaciens, Bacillus brevis, Bacillus circulans, Bacillus clausii, Bacillus coagulans, Bacillus lautus, Bacillus lentus, Bacillus licheniformis, Bacillus megaterium, Bacillus stearothermophilus, Bacillus subtilis, and Bacillus thuringiensis; or a Streptomyces cell, e.g., Streptomyces lividans and Streptomyces murinus, or gram negative bacteria such as E. coli and Pseudomonas sp. In a preferred embodiment, the bacterial host cell is a Bacillus lentus, Bacillus licheniformis, Bacillus stearothermophilus, or Bacillus subtilis cell. In another preferred embodiment, the Bacillus cell is an alkalophilic Bacillus.

Preferred non-natural eukaryotic host cells include CHO, myeloid, baby hampster kidney, COS, NSO, Hela and NIH323 cells, particularly, e.g., the monkey kidney CVI line transformed by SV40 (COS-7, ATCC CRL 1651); human embryonic kidney line (293, Graham et al. J. Gen Virol. 36:59 [1977]); baby hamster kidney cells (BHK, ATCC CCL 10); Chinese hamster ovary-cells-DHFR (CHO, Urlaub and Chasin, Proc. Natl. Acad. Sci. (USA) 77:4216, [1980]); mouse sertoli cells (TM4, Mather, Biol. Reprod. 23:243-251 [1980]); monkey kidney cells (CVI ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (hep G2, HB 8065); mouse mammary tumor (MMT 060562, ATCC CCL51); TRI cells (Mather et al., Annals N.Y. Acad. Sci 383:44-68 (1982)); human B cells (Daudi, ATCC CCL 213); human T cells (MOLT-4, ATCC CRL 1582); human macrophage cells (U-937, ATCC CRL 1593), single cell and filamentous yeast and fungi, and insect cells. The cells can be maintained according to standard methods well known to those of skill in the art (see, e.g., Freshney (1994) Culture of Animal Cells, A Manual of Basic Technique, (3d ed.) Wiley-Liss, New York; Kuchler et al. (1977) Biochemical Methods in Cell Culture and Virology, Kuchler, R. J., Dowden, Hutchinson and Ross, Inc. and the references cited therein). Cultured cell systems often will be in the form of monolayers of cells, although cell suspensions are also used, especially for commercial production.

Preferred features of expressing non-natural host cell lines include being an adventitious agent and/or infectious agent growing in virus and serum free medium, having fast growth and replication rates, and typically a small size and shear resistance. The cell lines also preferably have high but stable transcription and translation capacities, and are resistant to hypoxia. In certain circumstances, high transformation rates will be preferred.

Tissues of multicellular organisms, preferably agricultural plants and mammals, are also contemplated as suitable non-natural host cells of the present invention. For example, a nucleic acid encoding a protein native to a natural host tissue from one mammalian species may be translationally harmonized for, and introduced into, a non-natural host tissue that is another mammalian species. Similarly, a coding region from a species of one genus or kingdom can be harmonized for expression in, and introduced to, a non-natural host from another genus or kingdom. The tissue-types involved in such manipulations may be the same or different. For example, the natural host may be taken from one member of a group of preferred tissues, such as liver, spleen, lymphoid, smooth muscle, striated muscle, nerve tissue and adipose tissue, with the non-natural host being selected from a different tissue Including tissues that are not found in the natural host.

Examples of useful vertebrate tissues suitable as both natural and non-natural host cells include, but are not limited to, liver, kidney, spleen, bone marrow, thymus, heart, muscle, lung, brain, immune system (including lymphatic), testes, ovary, islet, intestinal, stomach, bone marrow, skin, bone, gall bladder, prostate, bladder, zygotes, embryos, and hematopoietic tissue. Useful vertebrate cell types include, but are not limited to, fibroblasts, epithelial cells, neuronal cells, germ cells (e.g., spermatocytes/spermatozoa and oocytes), stem cells, and follicular cells. Examples of plant tissues suitable as both natural and non-natural host cells include, e.g., leaf tissue, ovary tissue, stamen tissue, pistil tissue, root tissue, tubers, gametes, seeds, embryos, and the like.

Non-natural host cells may be transformed with integration cassettes using suitable means and cultured in conventional nutrient media modified as is appropriate for inducing promoters, selecting transformants or detecting expression. Suitable culture conditions for host cells, such as temperature and pH, are well known. The concentration of plasmid used for cellular transfection is preferably titrated to reduce the likelihood of expression in the same cell of multiple vectors encoding different affector RNA molecules. Freshney (Culture of Animal Cells, a Manual of Basic Technique, third edition Wiley-Liss, New York (1994)) and the references cited therein provides a general guide to the culture of cells. Transduced host cells are cultured by means well known in the art. See, also Kuchler et al. (1977) Biochemical Methods in Cell Culture and Virology, Kuchler, R. J., Dowden, Hutchinson and Ross, Inc. Mammalian cell systems often will be in the form of monolayers of cells, although mammalian cell suspensions are also used.

Transformed host cells expressing the protein encoded by the translationally-harmonized nucleic acid may be identified using assay techniques well known to those of skill in the art and include enzyme assays, colorimetric assays, immunological detection assays, spectrophotometric techniques and the like.

D. Quantitation Based on Expression Levels

The harmonized nucleic acids of the present invention are designed to provide expression of soluble recombinant proteins in a non-natural host that exceeds the expression rate of the native, non-harmonized, nucleic acid in the same host by at least 10%. Expression levels and rates may be determined using any method known to those of skill in the art as being compatible with the expression system and the recombinant protein being analyzed.

1. Immunological Assays

Quantitative immunological assays are well known, and include immunoprecipitation, Western blot analysis (immunoblotting), ELISA and fluorescence-activated cell sorting (FACS). Shapiro (2002) Practical Flow Cytometry (4th ed.) Wiley & Sons; ISBN: 0471411256; McCarthy and MacEy (eds. 2002) Cytometric Analysis of Cell Phenotype and Function Cambridge Univ. Press; ISBN: 0521660297; Givan (2001) Flow Cytometry: First Principles (2d ed.) Wiley-Liss; ISBN: 0471382248; Radbruch (ed. 2000) Flow Cytometry and Cell Sorting (2d. ed.; Springer Lab Manual) Springer-Verlag; ISBN: 3540656308; and Ormerod (ed. 2000) Flow Cytometry: A Practical Approach (3d. ed.) American Chemical Society; ISBN: 0199638241.

Antibodies directed to the expressed proteins can be identified and-obtained from a variety of sources, such as the MSRS catalog of antibodies (Aerie Corporation, Birmingham, Mich.), or can be prepared via conventional antibody generation methods. Methods for preparation of polyclonal antisera are taught in, for example, Ausubel, F. M. et al., Current Protocols in Molecular Biology, Volume 2, pp. 11.12.1-11.12.9, John Wiley & Sons, Inc., 1997. Preparation of monoclonal antibodies is taught in, for example, Ausubel, F. M. et al., Current Protocols in Molecular Biology, Volume 2, pp. 11.4.1-11.11.5, John Wiley & Sons, Inc., 1997.

Immunoprecipitation methods are standard in the art and can be found in, for example, Ausubel, F. M. et al., Current Protocols in Molecular Biology, Volume 2, pp. 10.16.1-10.16.11, John Wiley & Sons, Inc., 1998. Western blot (immunoblot) analysis is standard in the art and can be found at, for example, Ausubel, F. M. et al., Current Protocols in Molecular Biology, Volume 2, pp. 10.8.1-10.8.21, John Wiley & Sons, Inc., 1997. Enzyme-linked immunosorbent assays (ELISA) are standard in the art and can be found at, for example, Ausubel, F. M. et al., Current Protocols in Molecular Biology, Volume 2, pp. 11.2.1-11.2.22, John Wiley & Sons, Inc., 1991.

a. ELISA Assays

ELISA assays can be performed expressed proteins from both cellular or cell-free translation systems. For cell systems, the expressed protein is preferably a secreted protein. By way of example, secreted proteins are quantified by adding cell-depleted growth media to microtitre wells that contain immobilized antibodies that specifically bind the expressed protein. Typically a specific or selective reaction will be at least twice background signal or noise and more typically more than 10 to 100 times background. After sufficient time has elapsed for the immobilized antibodies to bind the reporter protein, the residual media is removed and a second antibody specific for a different epitope(s) of the expressed protein that is labeled with a detectable marker (e.g., a radiolabel, colored bead, enzyme or the like) is added. The immunocomplex formed is washed to remove excess labeled antibody and the label developed. The expression level of the integration cassette will be proportional to the amount of developed label present in the assay. (See, e.g., Harlow & Lane, Antibodies, A Laboratory Manual (1988), for a description of immunoassay formats and conditions that can be used to determine specific immunoreactivity).

b. FACS Assay

The fluorescence-activated cell sorter (FACS) can be used to both screen for successful transformation and quantitate expression levels. FACS analysis also lends itself to analysis of fusion proteins comprising a fluorescent label displayed on the cell surface, secreted, or expressed intracellularly, provided they are capable of producing a discernable fluorescent signal. If the expressed protein is secreted from a non-natural host cell, then the cells can be biotinylated and incubated with streptavidin conjugated to an antibody specific to the protein of interest (Manz et al., Proc. Natl. Acad. Sci. (USA) 92:1921 (1995)). Following incubation, the cells are placed in a high concentration of gelatin (or other polymer such as agarose or methylcellulose) to limit diffusion of the secreted protein. As protein is secreted by the cell, it is captured by the antibody bound to the cell surface. The presence of the protein is detected by a second antibody that is fluorescently labeled. For both secreted and membrane bound proteins, the cells can then be sorted according to their fluorescence signal. Fluorescent cells can then be isolated, expanded, and further enriched by FACS, limiting dilution, or other cell purification techniques known in the art.

Preferred fluorescent groups for use in fusion proteins of the invention are green fluorescent proteins (GFPs). GFPs are small proteins that can normally be expressed intracellularly without compromising cell viability. Fusion proteins expressed from translationally-harmonized nucleic acids and comprising GFP would be preferred over antibodies in FACS applications because such constructs do not have to be incubated with the fluorescent-tagged reagent and because there is no background due to nonspecific binding of an antibody conjugate. GFP also does not require any substrates or cofactors.

Another feature of FACS analysis is that expression levels can be determined coincidentally with transformation efficiency, and prior to clonal expansion. This saves time, and reagents as only cell candidates known to support expression levels meeting a minimum threshold value are for clonal expansion.

The level of expression of the protein is generally proportional to the fluorescent signal, regardless of the technique used. Moreover, the techniques relating to FACS lend themselves to automated, high throughput assays using microtitre plates and fluorescent signal plate readers.

Methods for conducting studies using FACS techniques may be found in, e.g., Shapiro (2002) Practical Flow Cytometry (4th ed.) Wiley & Sons; ISBN: 0471411256; McCarthy and MacEy (eds. 2002) Cytometric Analysis of Cell Phenotype and Function Cambridge Univ. Press; ISBN: 0521660297; Givan (2001) Flow Cytometry: First Principles (2d ed.) Wiley-Liss; ISBN: 0471382248; Radbruch (ed. 2000) Flow Cytometry and Cell Sorting (2d. ed.; Springer Lab Manual) Springer-Verlag; ISBN: 3540656308; and Ormerod (ed. 2000) Flow Cytometry: A Practical Approach (3d. ed.) American Chemical Society; ISBN: 0199638241.

c. Western Blot (Immunoblot) Analysis

Western blot analysis generally comprises separating expressed proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind the expressed protein(s). The antibodies may be directly labeled or alternatively may be subsequently detected using labeled antibodies (e.g., labeled sheep anti-mouse antibodies) that specifically bind to the anti-reporter antibodies.

4. Phenotypic Selection

In this embodiment for selection of transformants, cells can be selected based on a phenotype conferred by the expressed protein. Examples of phenotypes that can be selected for include proliferation, growth factor independent growth, colony formation, cellular differentiation (e.g., differentiation into a neuronal cell, muscle cell, epithelial cell, etc.), anchorage independent growth, activation of cellular factors (e.g., kinases, transcription factors, nucleases, etc.), gain or loss of cell—cell adhesion, migration, and cellular activation (e.g., resting versus activated T cells). Isolation of activated cells demonstrating a phenotype, such as those described above, is important because the activation/silencing of an endogenous gene by the integrated construct or reporter expression is presumably responsible for the observed cellular phenotype. Thus, the endogenous gene may be an important therapeutic drug or drug target for treating or inducing the observed phenotype.

IV. Isolating Proteins Expressed from Translationally-Harmonized Nucleic Acids

Proteins expressed using the translationally-harmonized nucleic acids of the present invention may be isolated from the translation systems used to express them using any technique, or combination of techniques, known to those of skill in the art. Suitable techniques include selective precipitation with such substances as ammonium sulfate; column chromatography using conventional matrices or those substituted with ligands (e.g., nickel-substituted nitroloacetic acid) that interact with encoded “affinity tags” (e.g., hexa histidine tags), immunopurification methods, and others (see, e.g., Scopes, Protein Purification: Principles and Practice (1982); U.S. Pat. No. 4,673,641; Ausubel et al., supra; and Sambrook et al., supra). A number of procedures can be employed to purify expressed fusion proteins of the invention. For example, expressed fusion proteins may be purified using immunoaffinity columns, or from growth-conditioned cell culture medium by immunoaffinity and ion exchange chromatography as described in Leonard et al., J. Biol. Chem. 265:10373-10382 (1990).

A. Solubility Fractionation

Often as an initial step, particularly if the protein mixture is complex, an initial salt fractionation can separate many of the unwanted translation system proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol includes adding saturated ammonium sulfate to a protein solution so that the resultant ammonium sulfate concentration is between 20-30%. This concentration will precipitate the most hydrophobic of proteins. The precipitate is then discarded (unless the protein of interest is hydrophobic) and ammonium sulfate is added to the supernatant to a concentration known to precipitate the protein of interest. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

B. Size Differential Filtration

The molecular weight of proteins expressed using the harmonized nucleic acids of the present invention can be used to isolate them from proteins of greater and lesser size using ultrafiltration through membranes of different pore size (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

C. Column Chromatography

Proteins expressed from translationally-harmonized nucleic acids of the present invention may also be separated from other proteins on the basis of size, net surface charge, hydrophobicity, and affinity for ligands. In addition, antibodies raised against proteins can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech or Merck).

All publications and patent applications cited in this specification are herein incorporated by reference as if each individual publication or patent application were specifically and individually indicated to be incorporated by reference.

Any of the specifically recited steps and/or functions described herein can be in the form of computer code and can be executed by one or more computational apparatuses such as one or more digitial computers. Any suitable computer readable media including magnetic, electronic, or optical disks or tapes, etc. can be used to store the computer code. The code may also be written in any suitable computer programming language including, for example, Fortran, Pascal, C, C++, etc. Some embodiments of the invention can be automatically performed without significant intervention on the part of a user.

Although the foregoing invention has been described in some detail by way of illustration and example for clarity and understanding, it will be readily apparent to one of ordinary skill in the art in light of the teachings of this invention that certain changes and modifications may be made thereto without departing from the spirit and scope of the appended claims.

As can be appreciated from the disclosure provided above, the present invention has a wide variety of applications. Accordingly, the following examples are offered for illustration purposes and are not intended to be construed as a limitation on the invention in any way. Those of skill in the art will readily recognize a variety of noncritical parameters that could be changed or modified to yield essentially similar results.

### EXAMPLES

The following MATERIALS AND METHODS were used in the examples that follow.

Materials and Methods:

Construction of Wild Type MSP1-42 (FVO)

Molecular cloning and bacterial transformations were performed as follows: MSP-1₄₂ fragment of FVO strain DNA was amplified by PCR from P. falciparum FVO genomic DNA by using the following primers:

| | | |
|---|---|---|
| FVO-PCR1; | 5′-GGGTCGGTACCATGGCAGTAACTC | (SEQ ID NO:1) |
| CTTCCGTAATTGAT-3′ | | |
| FVO-PCR2; | 5′GGATCAGATGCGGCCGCTTAACTGC | (SEQ ID NO:2) |
| AGAAAATACCATCGAAAAGTGGA-3′. | | |

The primers contained restriction sites for restriction endonucleases, NcoI and NotI, respectively. The vector for expression of wild type sequence MSP1-42 (FVO), pET(AT)FVO, was prepared by digesting pET(AT)PJMSP-1₄₂ (3D7) (Angov et. al. (2003) Molec. Biochem. Parasitol; in press) and the MSP-1₄₂ PCR fragment, with NcoI and NotI. The digested DNA's were purified by agarose gel extraction (QIAEXII, Qiagen, Chatsworth, Calif.), ligated with T4 DNA ligase (Roche Biochemicals) and transformed into E. coli BL21 DE3 (F⁻ ompT hsdS_{B} (r_{B}⁻m_{B}⁻) gal dcm (DE3) [Invitrogen, Carlsbad, Calif.] (Maniatis). Two clones were sequenced and found to be identical in this region to Genbank Accession number, L20092. Analysis of soluble expression levels from this clone yielded poor product yields and therefore eliminated this construct from further development.

Construction of Single Pause Site Mutant Expression Vector, pET(AT)FVO.A

The initial approach to improve soluble protein expression was to apply the harmonization approach in a highly restricted way, which was to identify areas of the protein that were likely to represent intradomain segments owing to the presence of clusters of infrequently used codons in the wild type gene. This restricted approach was taken in order to minimize the cost of producing synthetic DNA. The analysis revealed a single codon within an intradomain segment near the N-terminus of the protein that might benefit from harmonization. To prepare the expression vector, PET(AT)FVO.A, two overlapping oligonucleotides from within the wild type MSP-1₄₂ (FVO) gene sequence were designed to introduce a single synonymous codon substitution at codon #158 (codon ATC was changed to ATA) by using PCR primer-directed mutagenesis.

| | | |
|---|---|---|
| EA3, | 5′-TAAAAAATATATAAACGACAAAC-3′ | (SEQ ID NO:3) |
| EA5, | 5′-AAAAGGGAAGATATTTCTCATTT-3′ | (SEQ ID NO:4) |

The base pair changes away from wild-type sequence are underscored. In the first amplification, the 5′ end of the wild type MSP1₄₂ (FVO) template was amplified by PCR with the sense external primer FVO-PCR1 and the anti-sense internal primer EA5. In the second amplification, the 3′ end of the wild type MSP1₄₂ (FVO) template was amplified by PCR with the sense internal primer EA3 and the anti-sense external primer, FVO-PCR2. The two PCR products were purified by gel extraction using QIAEX II, mixed (1:1) and were used as the template for a final amplification to produce full gene MSP-1₄₂ using flanking primers FVO-PCR1 and FVO-PCR2. The final clone was prepared by digesting the vector DNA, pET(AT)PjMSP-1₄₂ (3D7), and insert DNA, with NcoI and NotI, and ligating together. The final pET(AT)FVO.A plasmid encodes 17 non-MSP1 amino acids including a hexa-histidine tag at the N-terminus of P. falciparum FVO strain MSP-1₄₂ sequence.

Construction of “Initiation Complex”—Harmonized MSP1-42 Expression Vector pET(K)FVO.B

The “initiation complex” harmonized MSP1-42 (FVO) clone was prepared by replacing the existing nucleotide sequence at the 5′-end of the MSP1-42 (FVO) gene sequence between restriction sites, KpnI and BspMI with annealed oligonucleotides that were designed to “harmonize” codon usage between P. falciparum usage and the E. coli host. To construct the “initiation complex” harmonized MSPI-42 (FVO), these two oligonucleotides pairs were synthesized, the sense strand,

| | | |
|---|---|---|
| EA485- | 5′-CGCAGTTACTCCATCTGTTATTGAT | (SEQ ID NO:5) |
| CDFVO, | AATATTCTTTCTAAAATTGAAA | |
| | ACGAATATGAGGTTTTATATTTAA-3′ | |
| EA493- | 5′-GGTTTTAAATATAAAACCTCATATT | (SEQ ID NO:6) |
| CDFVO, | CGTTTTCAATTTTAGAAAGAAT | |
| | ATTATCAATAACAGATGGAGTAACTGCG | |
| | GTAC-3′ | |

The oligonucleotides were designed, as reverse complimentary strands with overhanging restriction sites at each end such that direct ligation into vector, pET(AT)FVO.A, would replace the existing 5′- nucleotide sequence between the KpnI and BspMI sites. The oligonucleotides were annealed by adding 100 nmole/ml of each oligonucleotide, in a buffer containing 0.01 M Tris-HCl, pH 7.5, 0.1 M NaCl, and 0.001M EDTA. The mixture was heated to greater than 95° C. for 10 minutes and then removed from the heat source and allowed to cool to room temperature. To prepare the vector DNA, pET(AT)FVO.A, the vector was first restriction digested with BspMI such that the DNA was only restricted at the BspMI site located within the MSP1-42(FVO) DNA and not at the second BspMI site, located in the vector DNA sequence. Linearized DNA, 7.8 kb, was separated by electrophoreses on agarose gels and then gel purified using QIAEX II. Extracted, purified linear BspMI pET(AT)FVO.A DNA was then digested with KpnI to release the “foreign” sequence initiation complex, ˜100 bp. The vector DNA, containing KpnI and BspMI restricted ends was gel purified and then ligated with the KpnI and BspMI annealed oligonucleotides. The ligated DNA was transformed into E. coli host, BL21 DE3 and plated onto ampicillin plates. Colonies were screened for the correct insert by restriction digestion with NcoI. Restriction positive clones were tested for expression using the laboratory's standard bacterial culture and expression methods. The novel MSP1-42 (FVO) “initiation complex” harmonized clone, expressed from plasmid pET(AT)FVO.B, demonstrated a 10-15 fold increase in levels of soluble protein as compared to the MSP1-42 (FVO) single pause site mutant, clone pET(AT)FVO.A. To generate the final expression vector, the MSP1-42 (FVO) “initiation complex” harmonized insert DNA from plasmid DNA, pET(AT)FVO.B, was subcloned into the newly constructed antibiotic resistance-gene modified pET vector, pET(K), by restriction digestion with BamHI and NotI. The final expression vector for expression of MSP1-42(FVO) “initiation complex” harmonized is pET(K)FVO.B.

Construction of the Fully Harmonized Gene Expression Vector pET(K)FVO.C

To construct a synthetic gene for MSP1-42 (˜1100 nt PCR-based “extension-amplification” gene synthesis method was used. Briefly, consecutive pairs of sense and antisense oligonucleotides (each 50-60 nt) were synthesized, based on the fully harmonized sequence, to having 12-13 nt of complementary sequence on the 3′ ends; this allows the annealing of the oligonucleotide pair. Using PCR, the oligos were extended and amplified, and used in PCR reactions with subsequent amplified oligo pairs to “build” the synthetic gene. Because of the large size of the synthetic gene, four separate segments (“modules”) of ˜300 nt were created by using sequential PCR of the overlapping oligonucleotide pairs. These modules were selected so that the four segments could be joined by using three unique restriction enzyme sites (Hinc II, Bsrg I, Bst BI) present in the nucleotide sequence. To enable cloning into the pET(K) vector, an Nde I site was introduced just prior to the ATG initiation codon and tandem Not I and Xho I sites were included after the stop codon.

A series of PCR reactions yielded the four fragments. The first fragment begins with an Nde I site (before ATG codon) and ends with an Hinc II site. The second one starts with Hinc II and ends with a BsrG I site. The third one has BsrG I and Bst B I sites, and the last one had BstB I and Xho I sites (after the stop codon).

Each of the four fragments was generated separately and subcloned into a TA vector. In each instance, isolated transformants were selected and sequenced until a clone was identified as having the desired sequence and lacking mutations.

Each of the fragments was then purified from an agarose gel and ligated into a TA cloning vector, in sequence, by using T4 DNA ligase. For each step, competent host cells (TOP 10 supercompetent cells) were transformed with the ligation reaction and plated into antibiotic-selection plates and incubated at 37° C. Isolated colonies of transformants were grown to prepare plasmid DNA for agarose gel electrophoresis analysis. Several plasmids that appeared to contain insert were sequenced completely in order to select a clone without mutation. The final construct assembled from the four segments, pCR 2.1-MSP(1-42), was purified in sufficient quantities to allow transfer to the final pET(K) expression vector.

Purified pCR 2.1-MSP(1-42) vector was digested with Nde I and Xho I and the insert purified on a 1% agarose gel. The purified 1.1 kbp fragment was ligated by using T4 DNA ligase into the pET(K) expression vector which had been digested with Nde I and Xho I and purified on 1% agarose gel. Competent host cells (TOP 10 supercompetent cells) were transformed with the ligation reaction, plated into antibiotic-selection plates and incubated at 37° C. Isolated colonies of transformant were grown to prepare plasmid DNA for agarose gel electrophoresis analysis. Several plasmids that appeared to contain the final insert were sequenced in order to verify the integrity of the restriction sites.

Recombinant Protein Expression

For all constructions, E. coli B834 DE3 background cells were transformed with plasmids and were grown at 37° C. to an OD₆₀₀ of 0.5-0.8. The culture temperature was reduced from 37° C. to 25° C. prior to induction of protein expression with 0.1 mM IPTG. Induction was allowed to occur for 3.0 hours. At the end of the induction, cells were harvested by centrifugation at 27,666×g for 1 hr at 4° C. and the cell paste was stored at −80° C.

Partial Protein Purification for Comparison of Expression Levels.

2-3 g cells were suspended in 20 ml 10 mM sodium phosphate, 50 mM NaCl, 10 mM imidazole, pH 6.2. The sample was lysed by using a microfluidizer and Tween 80 was added to a final concentration of 1%, and NaCl to a final concentration of 500 mM. The sample was stirred for 15 ml a 0-4° C., centrifuged for 30 min at 27,000 g at 0-4° C. and the supemate collected. The proteins were purified partially by chromatography on Ni⁺² NTA Superflow (Qiagen, Chatsworth, Calif.). A 700 ul column was equilibrated with 0.01M sodium chloride, pH 6.2, 500 mM sodium chloride, 0.01 M imidazole (Ni-buffer) and 0.5% Tween 80. The sample was applied and the column washed with 10 ml of 10 mM sodium phosphate, pH 6.2, 75 mM sodium chloride, 0.02 M imidazole. The pH was the changed by washing with 10 ml 10 mM sodium phosphate buffer, pH 8.0, 75 mM sodium chloride, 0.02 M imidazole. The proteins were eluted in 3.5 ml of 10 mM sodium phosphate, pH 8.0, 75 mM sodium chloride, 160 mM imidazole and 0.2% Tween 80.

Partial Purification of E. coli Expressed Fully Harmonized Gene MSP-1₄₂ (FVO) for Investigation of Solubility.

Cell paste was lysed in buffer containing phosphate buffered saline, pH 7.4 containing 0.01 M imidazole and 50 U/ml benzonase. Following cell lyses by microfluidization, the lysate was either incubated in the presence or absence of the non-ionic detergent, Tween 80 (1.0%, v/v) on ice for 30 minutes with stirring, prior to centrifugation at 27,666×g for 1 hr at 4° C. This clarified lysate was centrifuged at 100,000 g for 1 hour to show that the protein is expressed in soluble form in the cell cytoplasm or it was applied to a Ni⁺² NTA superflow resin for partial purification.

SDS-PAGE and Immunoblotting.

Proteins were separated by Tris-Glycine SDS-PAGE under non-reducing or reducing (10% 2-mercaptoethanol) conditions. Total protein was detection by Coomassie Brilliant Blue R-250 (Bio-Rad Laboratories, Hercules, Calif.) staining and immunoblotting are as previously described (3D7 manuscript) Nitrocellulose membranes were probed with either polyclonal mouse anti-FVO MSP-142 antibodies (a gift from Dr. Sanjai Kumar, FDA, Bethesda, Md.), polyclonal rabbit anti-E. coli antibodies (GSK) or mouse mAbs diluted into PBS, pH 7.4 containing 0.1% Tween 20. The mAbs used for evaluation of proper epitope structure included 2.2 (McBride et al, 1987, Mol. Biochem. Parasitol., 23, 71-84; Hall et al, 1983, Mol. Biochem. Parasitol, 7, 247-65), 12.8 (McBride, 1987, supra; Blackman et al, 1990, J. Exp. Med., 172, 379-82), 7.5 (McBride, 1987, supra; Hall et al, 1983, supra), 12.10 (McBride, 1987, supra; Blackman et al, 1990, supra), 5.2 (Chang et al, 1988, Exp. Parasitol., 67, 1-11).

### Example 1

In a studies design to produce a potentially important malarial antigen, we found that the levels of soluble MSP1-42 (FVO) protein obtained following induction of BL21 DE3 cells expressing the wild type gene sequence, pET(AT)FVO were negligible and insufficient to advance for further process development. Rather than changing to a new expression system, such a Pichia, or baculovirus, we chose to try to overcome this problem owing to the advantages that E. coli offers, especially with respect to expression of non-glycosylated protein. Our initial thinking was that it might be important to preserve ribosomal pausing (i.e., attenuate translation) at certain times to allow for protein folding. We thought that we might achieve this by analyzing the target gene to reveal clusters of low abundance codons and changing those codons if necessary (“harmonizing”) so that they would correspond to codons of low abundance in the expression host (in this case E. coli). For the first approach to codon harmonization, we used, as reference materials, codon frequency tables for P. falciparum (Saul A & Battistutta D. Codon usage in Plasmodium falciparum. Mol Biochem Parasitol 1988; 27:35-42.) and E. coli (Data Reference Set, Volume 3: Data Files, Genetics Computer Group, Sequence Analysis Software Package). The entire codon usage data set for both organisms is presented in table 1. We evaluated consecutive codons as rolling triplets along the range of amino acids of interest, paying special attention to the patterns associated with domain segments, which separate minimal domain structures, i.e. alpha helices, beta pleated sheets. Within interdomain segments, the amino acid content is restricted to about half of the common amino acids and their corresponding codons tend to be used infrequently, indicating that translation proceeds slowly in these regions. This slowdown in translation within interdomain segments may allow nascent protein to complete the folding of one domain prior to initiating synthesis of the next.

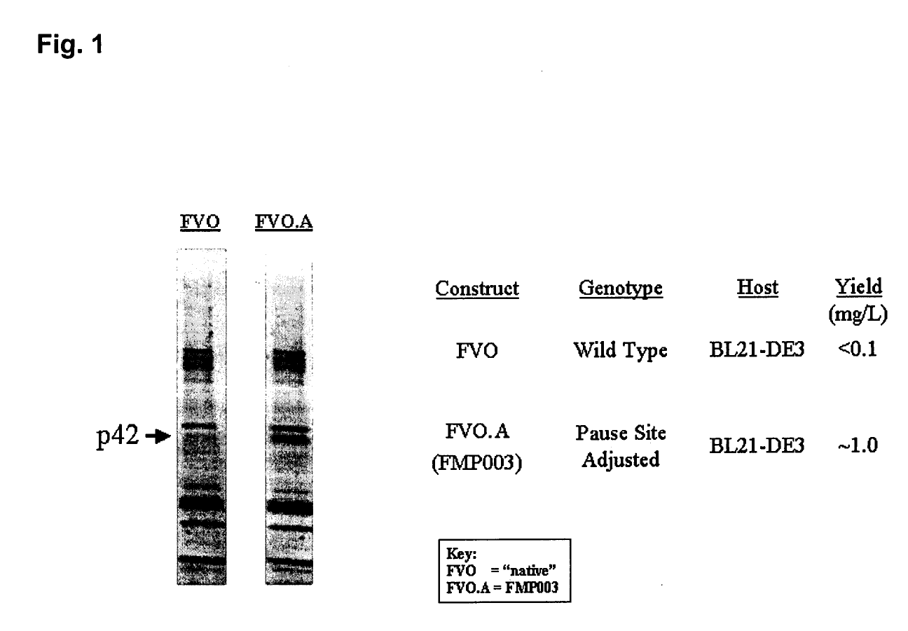

Using this method of locating infrequently used codons we predicted putative translation pause sites (low frequency used codons in P. falciparum). We first identified a single amino acid substitution within the translated native sequence, residue #158, at which harmonization was needed to create a codon having a corresponding low relative codon usage for expression in E. coli. The Coomassie Blue stained gels shown in FIG. 1 compares partially purified wild type vs. single pause site mutant MSP1-42 (FVO), FMPOO3. The relative increase in soluble MSP1-42 expression is approximately 10 fold above wild type. At that time we recognized that “fully harmonizing” a gene might be the best strategy; we took this initial “limited” approach owing to the expense associated with making synthetic genes.

### Example 2

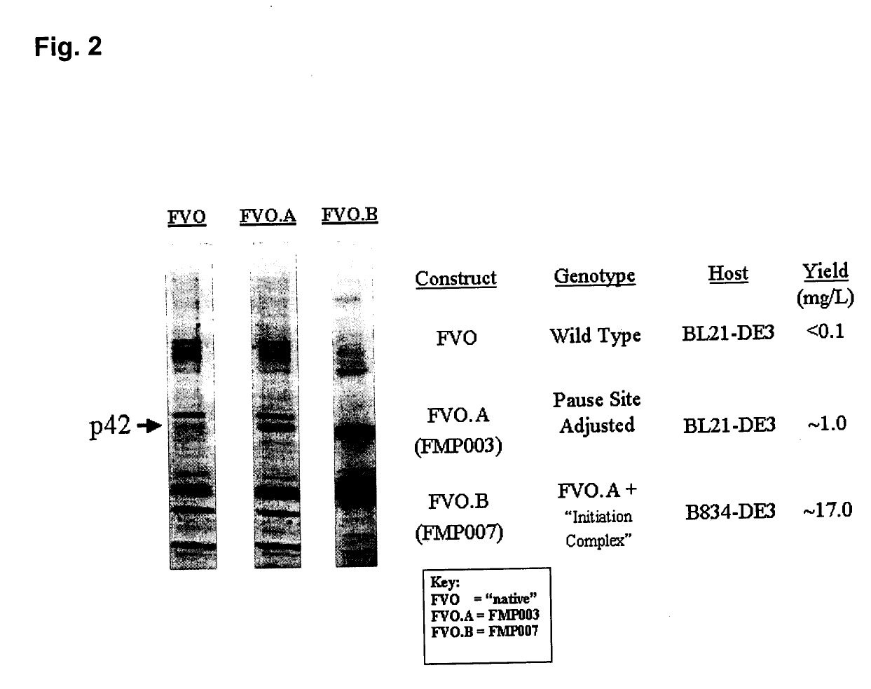

While the FMP003 product was estimated to yield approximately 10 fold more soluble MSP1-42 than wild type sequence, the final product yield, at 1 mg/L, was still insufficient for advanced development where target product yields are in the range of 100 mg/L. Therefore, for the second approach, E. coli codons were harmonized to P. falciparum codons with the objective of preserving high and low relative codon usage rates in the region of the initiation complex. A hypothesis is that stabilizing the interaction of the ribosome on the initiation complex might lead to increased levels of translation, or that translation from a properly harmonized initiation complex might allow for the initiation of proper protein folding. Again, using existing codon frequency tables referred to above, we applied the same process more broadly to reveal all codons in the “initiation complex” region that were mismatched for codon usage frequency between the natural gene and the expression host. Five synonymous codon replacements were made, resulting in an additional 10-15 fold increase in soluble product when compared to FMP003. The estimated product yield for FMP007 is 15 mg/L based on small-scale chromatography. The levels of final product produced are substantially above the wild type MSP1-42 and the FMP003 product (See FIG. 2).

### Example 3

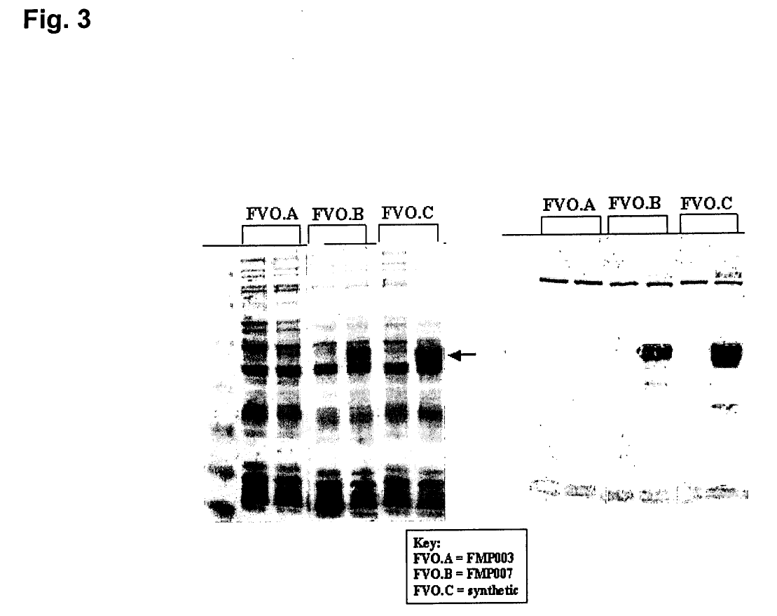

In light of the improvement in yield of FMP007 compared with FMP003, we decided to design a fully harmonized gene for expression. This decision was supported by our results from the fully-harmonized gene for the malaria antigen, LSA-NRC (see below, Example 4), which resulted in bacterial expression levels of 30-50% of the total protein in the cell lysate, all of which was soluble in the host cell cytoplasm. As described above, using the referenced tables of codon usage, we synthesized a fully-harmonized gene for MSP1-42 and carried out expression studies in E. coli. (See FIG. 3).

In this final approach, E. coli codons were harmonized to P. falciparum codons with the objective of preserving all high and low codon usage rates throughout the gene sequence. This effort resulted in additional 10-fold increase in the yield of protein from the fully harmonized gene over that of FMP007 and at least half of the protein was soluble in the host cell cytoplasm (FIG. 3). The remarkable increase in soluble protein expression (˜100 increase) appears to underscore the importance of maintaining the pattern of relative codon usage seen in the natural host when carrying out heterologous protein expression.

### Example 4

In a study of a different malarial antigen, expression, purification and characterization of a recombinant P. falciparum LSA-1 gene construct, lsa-nrc, was undertaken with the aim of producing GMP grade protein for development as a pre-erythrocytic vaccine. The LSA-NRC protein contains the highly conserved N- and C-terminal regions and two 17 amino acid repeat units of the 3D7 sequence of the P. falciparum LSA-1 protein. Two distinct approaches were undertaken to improve the protein yield by genetically re-engineering the gene sequence from the original P. falciparum sequence. In the first approach the gene construct was designed using codons with the highest relative codon usage in E. coli (class 2 proteins), i.e. the gene was “optimized” using the conventional approach to use preferred codons. In the second approach, the gene construct was designed by translationally “harmonizing” codon usage, based on by existing codon frequency tables for P. falciparum and E. colito more closely match the relative codon usage in P. falciparum. An comparison of how each approach affects codon selection is shown in the Table 2.

TABLE 2

| | | Non-natural | Relative codon | Non-natural | Relative codon |
|---|---|---|---|---|---|
| Natural host | Relative codon | Codon | Usage (%) | Codon | Usage (%) |
| Codon | Usage (%) | (E. coli c1.2) | (E. coli c1.2) | (E. coli c1.2) | (E. coli c1.2) |
| (P. falciparum) | (P. falciparum) | “optimized” | “optimized” | “harmonized” | “harmonized” |
| AAC | −73 | AAC | 65 | AAT | −65 |
| TTG | −18 | CTG | 359 | CTC | −50 |
| AGA | 263 | CGT | 335 | CGC | 98 |

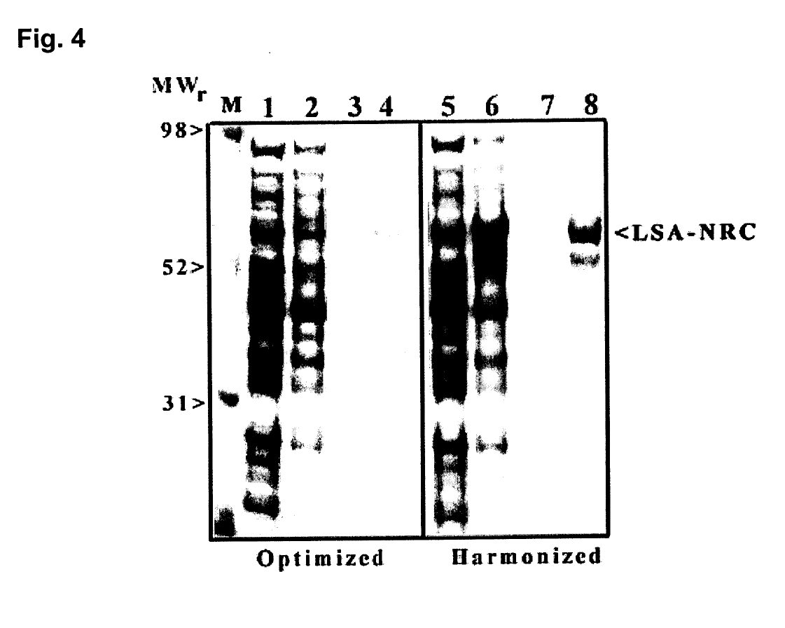

When the native lsa-nrc gene was expressed, very little product was observed, owing in part to the reduced use of “preferred” E. coli codons in the natural malarial gene. “Optimization” of the gene was done, using the preferred codons for “class 2” E. coli proteins, resulting in increased protein production (FIG. 2); however, the resulting protein was found to be insoluble (data not shown). When a synthetic gene for lsa-nrc gene was engineered for heterologous expression by “harmonizing” codon usage (lsa-nrc/H) significantly more recombinant protein was produced than using highest frequency E. coli (lsa nrc/E) codons; furthermore, virtually all of the produced protein was soluble. A demonstration of the high-level expression of protein is shown in FIG. 4.

The preceding example serves to illustrate that use of a most-preferred codon at each residue for that amino acid may in fact lead to improper folding of the protein in contrast to the use of codons whose relative usage frequency is “matched” to that seen in the natural host.

What is claimed is:

1. A method of increasing soluble levels of a heterologous protein expressed in a non-natural host by selective translational attenuation, the method comprising:
a) synthesizing a translationally-harmonized nucleic acid comprising a coding region consisting of at least 60 contiguous codons and having a nucleotide sequence at least 65% identical to a contiguous codon sequence native to a natural host wherein the coding region is translationally-harmonized by substituting each of at least 3 codons of the native contiguous codon sequence with a synonymous codon that has a lower usage frequency in the non-natural host, where the codons being replaced;
(i) are within 10 codons of a codon in the native codon sequence that has a low or intermediate codon usage frequency in the natural host; and,
(ii) are of intermediate or high usage frequency in the non-natural host;
b) introducing the harmonized nucleic acid into the non-natural host cell to generate a recombinant host; and,
c) culturing the non-natural host under conditions that permit the soluble level of the heterologous protein to exceed by more than 10% the soluble level of that protein when expressed from a non-harmonized nucleic acid in the non-natural host.

2. The method of claim 1, wherein the nucleotide sequence of the 60 contiguous codons is at least 85% identical to the nucleotide sequence of the native contiguous codon sequence.

3. The method of claim 1, wherein the coding region is translationally-harmonized by substituting each of at least 6 codons of the native contiguous codon sequence with a synonymous codon of the non-natural host.

4. The method of claim 1, wherein the codons being replaced are within 6 codons of a codon in the native codon sequence that has a low or intermediate codon usage frequency in the natural host.

5. The method of claim 1, wherein the codons being replaced are within 3 codons of a codon in the native codon sequence that has a low or intermediate codon usage frequency in the natural host.

6. The method of claim 1, wherein the translationally-harmonized nucleic acid further comprises expression control sequences operably-linked to the coding region.

7. The method of claim 1, wherein the heterologous protein is a fusion protein and the translationally-harmonized nucleic acid further comprises a second coding region in-frame with the first coding region.

8. The method of claim 7, wherein the translationally-harmonized nucleic acid further comprises a nucleotide sequence encoding a peptide linker in-frame with and located between the coding region and the second coding region.

9. The method of claim 7, wherein the second coding region encodes an affinity tag.

10. The method of claim 7, wherein the second coding region is translationally harmonized.

11. The method of claim 1, wherein the non-natural host is a bacterial cell.

12. The method of claim 1, wherein the non-natural host is a fungal cell.

13. The method of claim 1, wherein the non-natural host is an insect cell.

14. The method of claim 1, wherein the non-natural host is a plant cell.

15. The method of claim 1, wherein the natural host is a mammalian tissue cell and the non-natural host is a different mammalian tissue cell.

16. The method of claim 1, wherein the natural host is a first cell normally residing in a first mammalian species and the non-natural host is a second cell normally residing in a second mammalian species.

17. The method of claim 16, wherein the first cell and the second cell are from the same tissue type selected from the group consisting of liver, spleen, lymphoid, smooth muscle, striated muscle, nerve tissue and adipose tissue.

18. The method of claim 1, wherein the coding region encodes a mammalian protein.

19. The method of claim 18, wherein the coding region encodes a mammalian hormone.

20. The method of claim 1, wherein the coding region encodes a neuropeptide.

21. The method of claim 1, wherein the coding region encodes an antibody.

22. The method of claim 1, wherein the coding region encodes an antimetabolite.

23. The method of claim 1, wherein the coding region encodes an antibiotic.

24. The method of claim 1, further comprising isolating the heterologous protein.

25. The method of claim 24, wherein the heterologous protein further comprises a secretory sequence and isolating the heterologous protein comprises centrifuging the non-natural host culture.

26. A translationally-harmonized nucleic acid comprising a coding region for a recombinant protein, the coding region consisting of at least 60 contiguous codons and having a nucleotide sequence at least 65% identical to a contiguous codon sequence native to a natural host wherein the coding region is translationally-harmonized by substituting each of at least 3 codons of the native contiguous codon sequence with a synonymous codon that has a lower usage frequency in the non-natural host, where the codons being replaced;
(a) are within 10 codons of a codon in the native codon sequence that has a low or intermediate codon usage frequency in the natural host; and,
(b) are of intermediate or high usage frequency in the non-natural host.

27. The nucleic acid of claim 26, wherein the nucleotide sequence of the 60 contiguous codons is at least 85% identical to the nucleotide sequence of the native contiguous codon sequence.

28. The nucleic acid of claim 26, wherein the coding region is translationally-harmonized by substituting each of at least 6 codons of the native contiguous codon sequence with a synonymous codon of the non-natural host.

29. The nucleic acid of claim 26, wherein the codons being replaced are within 6 codons of a codon in the native codon sequence that has a low or intermediate codon usage frequency in the natural host.

30. The nucleic acid of claim 26, wherein the codons being replaced are within 3 codons of a codon in the native codon sequence that has a low or intermediate codon usage frequency in the natural host.

31. The nucleic acid of claim 26, further comprising expression control sequences operably-linked to the coding region.

32. The nucleic acid of claim 26, wherein the heterologous protein is a fusion protein and the translationally-harmonized nucleic acid further comprises a second coding region in-frame with the first coding region.

33. The nucleic acid of claim 32, further comprising a nucleotide sequence encoding a peptide linker in-frame with and located between the coding region and the second coding region.

34. The nucleic acid of claim 32, wherein the second coding region encodes an affinity tag.

35. The nucleic acid of claim 32, wherein the second coding region is translationally harmonized.

36. The nucleic acid of claim 26, wherein the non-natural host is a bacterial cell.

37. The nucleic acid of claim 26, wherein the non-natural host is a fungal cell.

38. The nucleic acid of claim 26, wherein the non-natural host is an insect cell.

39. The nucleic acid of claim 26, wherein the non-natural host is a plant cell.

40. The nucleic acid of claim 26, wherein the natural host is a mammalian tissue cell and the non-natural host is a different mammalian tissue cell.

41. The nucleic acid of claim 26, wherein the natural host is a first cell normally residing in a first mammalian species and the non-natural host is a second cell normally residing in a second mammalian species.

42. The nucleic acid of claim 41, wherein the first cell and the second cell are from the same tissue type selected from the group consisting of liver, spleen, lymphoid, smooth muscle, striated muscle, nerve tissue and adipose tissue.

43. The nucleic acid of claim 26, wherein the coding region encodes a mammalian protein.

44. The nucleic acid of claim 43, wherein the coding region encodes a mammalian hormone.

45. The nucleic acid of claim 26, wherein the coding region encodes a neuropeptide.

46. The nucleic acid of claim 26, wherein the coding region encodes an antibody.

47. The nucleic acid of claim 26, wherein the coding region encodes an antimetabolite.

48. The nucleic acid of claim 26, wherein the coding region encodes an antibiotic.

49. A recombinant cell comprising an expression system, including a translationally-harmonized nucleic acid comprising a coding region for a recombinant protein, the coding region consisting of at least 60 contiguous codons and having a nucleotide sequence at least 65% identical to a contiguous codon sequence native to a natural host wherein the coding region is translationally-harmonized by substituting each of at least 3 codons of the native contiguous codon sequence with a synonymous codon that has a lower usage frequency in the recombinant cell, where the codons being replaced;
(a) are within 10 codons of a codon in the native codon sequence that has a low or intermediate codon usage frequency in the natural host; and,
(b) are of intermediate or high usage frequency in the recombinant cell;
wherein the recombinant cell produces a soluble level of the heterologous protein exceeding the soluble level of that protein when expressed from a non-harmonized nucleic acid in the recombinant cell by more than 10%.

50. The recombinant cell of claim 49, wherein the nucleotide sequence of the 60 contiguous codons is at least 85% identical to the nucleotide sequence of the native contiguous codon sequence.

51. The recombinant cell of claim 49, wherein the coding region is translationally-harmonized by substituting each of at least 6 codons of the native contiguous codon sequence with a synonymous codon of the recombinant cell.

52. The recombinant cell of claim 49, wherein the codons being replaced are within 6 codons of a codon in the native codon sequence that has a low or intermediate codon usage frequency in the natural host.

53. The recombinant cell of claim 49, wherein the codons being replaced are within 3 codons of a codon in the native codon sequence that has a low or intermediate codon usage frequency in the natural host.

54. The recombinant cell of claim 49, wherein the translationally-harmonized nucleic acid further comprises expression control sequences operably-linked to the coding region.

55. The recombinant cell of claim 49, wherein the heterologous protein is a fusion protein and the translationally-harmonized nucleic acid further comprises a second coding region in-frame with the first coding region.

56. The recombinant cell of claim 55, wherein the second coding region encodes an affinity tag.

57. The recombinant cell of claim 55, wherein the second coding region is translationally harmonized.

58. The recombinant cell of claim 55, wherein the translationally-harmonized nucleic acid further comprises a nucleotide sequence encoding a peptide linker in-frame with and located between the coding region and the second coding region.

59. The recombinant cell of claim 49, wherein the recombinant cell is a bacterial cell.

60. The recombinant cell of claim 49, wherein the recombinant cell is a fungal cell.

61. The recombinant cell of claim 49, wherein the recombinant cell is an insect cell.

62. The recombinant cell of claim 49, wherein the recombinant cell is a plant cell.

63. The recombinant cell of claim 49, wherein the natural host is a mammalian tissue cell and the recombinant cell is a different mammalian tissue cell.

64. The recombinant cell of claim 49, wherein the natural host is a first cell normally residing in a first mammalian species and the recombinant cell is a second cell normally residing in a second mammalian species.

65. The recombinant cell of claim 64, wherein the first cell and the second cell are from the same tissue type selected from the group consisting of liver, spleen, lymphoid, smooth muscle, striated muscle, nerve tissue and adipose tissue.

66. The recombinant cell of claim 49, wherein the coding region encodes a mammalian protein.

67. The recombinant cell of claim 66, wherein the coding region encodes a mammalian hormone.

68. The recombinant cell of claim 49, wherein the coding region encodes a neuropeptide.

69. The recombinant cell of claim 49, wherein the coding region encodes an antibody.

70. The recombinant cell of claim 49, wherein the coding region encodes an antimetabolite.

71. The recombinant cell of claim 49, wherein the coding region encodes an antibiotic.

72. A computer readable medium comprising:
a) code for a first set of instructions for identifying at least three codons in a first set of at least 60 statically ordered codons, wherein the identified codons have a low or intermediate codon usage in a first codon usage data set;
b) code for a second set of instructions for substituting each of the identified codons with a synonymous codon having a low or intermediate codon usage in a second codon usage data set, thereby generating a second set of at least 60 statically ordered codons;
wherein the first set of at least 60 codons is representative of a nucleic acid sequence of a natural host; the first codon usage data set contains data identifying the frequency of codon usage in expressed nucleic acid transcripts of the natural host for each synonymous codon corresponding to each natural amino acid; the second codon usage data set contains data identifying the frequency of codon usage in expressed nucleic acid transcripts of a non-natural host for each synonymous codon corresponding to each natural amino acid; and, the second set of at least 60 codons is representative of a nucleic acid translationally-harmonized for expression in the non-natural host.

73. The computer readable medium of claim 72, wherein the first set of instructions identify at least three codons in a first set of at least 60 statically ordered codons.

74. The computer readable medium of claim 72, wherein the non-natural host is a bacterial cell.

75. The computer readable medium of claim 72, wherein the non-natural host is a fungal cell.

76. The computer readable medium of claim 72, wherein the non-natural host is an insect cell.

77. The computer readable medium of claim 72, wherein the non-natural host is a plant cell.

78. The computer readable medium of claim 72, wherein the natural host is a mammalian tissue cell and the non-natural host is a different mammalian tissue cell.

79. The computer readable medium of claim 72, wherein the natural host is a first cell normally residing in a first mammalian species and the non-natural host is a second cell normally residing in a second mammalian species.

80. The computer readable medium of claim 79, wherein the first cell and the second cell are from the same tissue type selected from the group consisting of liver, spleen, lymphoid, smooth muscle, striated muscle, nerve tissue and adipose tissue.

81. The computer readable medium of claim 72, wherein the at least 60 statically ordered codons encodes a mammalian protein.

82. The computer readable medium of claim 81, wherein the at least 60 statically ordered codons encodes a mammalian hormone.

83. The computer readable medium of claim 72, wherein the at least 60 statically ordered codons encodes a neuropeptide.

84. The computer readable medium of claim 72, wherein the at least 60 statically ordered codons encodes an antibody.

85. The computer readable medium of claim 72, wherein the at least 60 statically ordered codons encodes an antimetabolite.

86. The computer readable medium of claim 72, wherein the at least 60 statically ordered codons encodes an antibiotic.
